(19) United States
(12) Patent Application Publication
(10) Pub. No.: US 2003/0140369 A1
(43) Pub. Date: Jul. 24, 2003

(54) Novel defense induced multi-drug resistance genes and uses thereof

(75) Inventor: Carl R. Simmons, Des Moines, IA (US)

Correspondence Address: ALSTON & BIRD LLP, PIONEER HI-BRED INTERNATIONAL, INC., BANK OF AMERICA PLAZA, 101 SOUTH TYRON STREET, SUITE 4000, CHARLOTTE, NC, 28280-4000, US

(73) Assignee: Pioneer Hi-Bred International, Inc.

(21) Appl. No.: 10/237,852

(22) Filed: Sep. 6, 2002

Related U.S. Application Data
(63) Continuation-in-part of application No. 09/790,099, filed on Feb. 21, 2001.
(60) Provisional application No. 60/185,958, filed on Feb. 29, 2000.

(51) Int. Cl. A01H 5/00; C07H 21/04; C12N 9/00; C12P 21/02; C12N 5/04

(52) U.S. Cl. 800/279; 536/23.6; 435/419; 435/320.1; 435/69.1; 435/183

(57) ABSTRACT

The invention provides isolated defense induced plant subfamily multi-drug resistance gene nucleic acids and their encoded proteins. The present invention provides methods and compositions relating to altering these defense induced multi-drug resistance gene levels in plants to improve resistance to plant pathogens. The invention further provides recombinant expression cassettes, host cells, transgenic plants, and antibody compositions.

### CROSS-REFERENCE TO RELATED APPLICATIONS

This application is a continuation-in-part of U.S. application Ser. No. 09/790,099, filed on Feb. 21, 2001, which claims the benefit of U.S. Provisional Application No. 60/185,958, filed on Feb. 29, 2000, both of which are hereby incorporated by reference.

### FIELD OF THE INVENTION

The present invention relates generally to plant molecular biology. More specifically, it relates to nucleic acids and methods for modulating their expression in plants.

### BACKGROUND OF THE INVENTION

In the past improving disease resistance or tolerance in crop plants typically involved elaborate breeding to incorporate natural resistance mechanisms into elite breeding material. The sources of this natural resistance were often otherwise undesirable plant materials, and so extensive backcrossing and introgression was needed to recreate the desired background with the disease resistance. Sometimes even this was not obtained, as the resistance mechanism(s) were polygenic. In short, improving disease resistance by conventional breeding is expensive in both time and money and is of uncertain results.

One mechanism, among the various mechanisms, plants use for defence against pathogenic organisms is the constitutive or inducible expression of proteins with antimicrobial function (Agrios, Plant Pathology, 4^{th} Edition, Academic Press, San Diego, Calif., p 635 (1997)). These proteins are generally referred to as pathogenesis-related (PR) proteins, and there are now at least 14 classes known (Hammond-Kosack and Jones, Responses to Plant Pathogens. In Biochemistry and Molecular Biology of Plants, Buchanan, Guissem, & Jones, Eds. American Society of Plant Physiologists, Rockville Md. pp 1102-1156 (2000)). Where known, the biochemical mechanisms of PR proteins appear to be varied. PR genes tend to be coordinately induced following pathogen attack, and they are generally thought to be major determinants of resistance only collectively, with single genes usually being minor determinants. Studies of PR protein expression to date have largely relied on assaying one or a few genes at a time. RNA and protein profiling technologies, used in conjunction with expanded gene sequence databanks, now allow for thousands of gene expression changes to be assayed in a single experiment, providing the opportunity for identifying new PR proteins.

One such group of these PR proteins is the antibiotic efflux transporters, which belong to several diverse classes. Among the various classes of antibiotic efflux pumps, a large group is the major facilitator superfamily (MFS), which to date have been predominantly studied in bacteria (Marger and Saier, Trends in Biochemical Science 18: 13-20 (1993)). The mechanism of MFS transport is thought to typically operate via proton motive force, with the incoming proton exchanged for the efflux compound. The role of plant MFS transporters is just now coming to light, with the identification of members involved in transport of sugars (Lemoine, Biochimica et Biophysica Acta 1465: 246-262 (2000); Quirino et al., Plant Mol Biol 46: 447-457 (2001)), and of nitrate (Trueman et al., Gene 175: 223-231 (1996)). Nonetheless their role in plant defense has apparently not been reported. However, there are now reports that plant pathogenic fungi utilize MFS antiporters to expel their own toxins, thus rendering themselves resistant, while exposing toxins to the plant. These include the CFP protein that effluxes the polyketide cercosporin produced by Cercospora kikuchii, a soybean pathogen (Callahan et al., Mol Plant Microbe In 12: 901-910 (1999)), the ToxA protein that effluxes the cyclic tetrapeptide HC-toxin produced by Cochliobolus carbonum, a maize pathogen (Pitkin et al., Microbiology 142: 1557-1565 (2000)), and the TRI12 trichothecen efflux pump from Fusarium sporotrichioides (Alexander et al., Plant Phys 79: 843-847 (1999)).

Consequently, pathogen MFS proteins are now thought to control the exchange of toxins governing plant-pathogen interactions, but the role of the plant MFS counterparts remains largely unknown. MFS proteins can also have potassium efflux and re-uptake function, which may also relate to a defense role, as potassium efflux is a well-known phenomenon of plant responses to pathogens, but for which specific transporters is not yet known.

What is needed in the art is a means to improve plant disease resistance, particularly in crop plants such as cereals. The present invention provides this and other advantages through the use of plant MFS proteins.

### SUMMARY OF THE INVENTION

The present invention provides nucleic acids and proteins relating to defense induced genes (DIG) in maize, rice, and wheat. Further, the present invention provides transgenic plants comprising the nucleic acids of the present invention, and methods for modulating, in a transgenic plant, expression of the nucleic acids of the present invention.

Therefore, one aspect the present invention relates to an isolated nucleic acid comprising a member selected from the group consisting of (a) a polynucleotide having a specified sequence identity to a polynucleotide encoding a polypeptide of the present invention; (b) a polynucleotide which is complementary to the polynucleotide of (a); and, (c) a polynucleotide comprising a specified number of contiguous nucleotides from a polynucleotide of (a) or (b). The isolated nucleic acid can be DNA.

In other aspects the present invention relates to: 1) recombinant expression cassettes, comprising a nucleic acid of the present invention operably linked to a promoter, 2) a host cell into which the recombinant expression cassette has been introduced, and 3) a transgenic plant comprising the recombinant expression cassette. The host cell and plant are optionally a maize cell or maize plant, respectively.

### BRIEF DESCRIPTION OF THE DRAWINGS

Having thus described the invention in general terms, reference will now be made to the accompanying drawing, which is not necessarily drawn to scale, and wherein:

FIG. 1 shows a Kyte-Doolittle hydrophobicity comparison between the maize gene p0018.chsth71r peptide (SEQ ID NO: 2; protein 1, profile marked A) and that of a multidrug resistance protein from Pasteurella haemolytica (protein 2, profile marked B).

### DETAILED DESCRIPTION OF THE INVENTION

Unless otherwise stated, the polynucleotide and polypeptide sequences identified in Table 1 represent polynucleotides and polypeptides of the present invention. Table 1 cross-references these polynucleotide and polypeptides to their gene name and internal database identification number. A nucleic acid of the present invention comprises a polynucleotide of the present invention. A protein of the present invention comprises a polypeptide of the present invention.

TABLE 1

| | | Polynucleotide | Polypeptide |
|---|---|---|---|
| Gene Name | Database ID NO: | SEQ ID NO: | SEQ ID NO: |
| Defense Induced | p0018.chsth71r | 1 | 2 |
| Gene (DIG) (Maize) | | | |
| DIG (Maize) | p0032.crcbg26r | 3 | |
| DIG (Maize) | p0085.cscan24r | 4 | |
| DIG (Maize) | p0095.cwsbh58r | 5 | |
| DIG (Maize) | p0126.cnleh06r | 6 | |
| DIG (Rice) | rds1f.pk002.a8 | 7 | |
| DIG (Rice) | rls24.pk0021.d7 | 8 | |
| DIG (Wheat) | wre1n.pk0130.d1 | 9 | |

The present invention provides utility in such exemplary applications as modulating resistance or tolerance to known crop plant pathogens. In some embodiments resistance is increased. Pathogens to which the invention can be applied include fungi, bacteria, viruses, and other microbes. Pathogens also include nematodes and insects. Further, the present invention modulates abiotic stress related diseases caused by heat, drought, cold, reactive oxygen species and radiation. This invention especially pertains to modulating resistance to fungal pathogens. Cereal crops, such as maize, wheat, or rice, are exemplary crops to which the invention may be applied.

Library Construction

Table 2 references various DIG clones and provides their homology to reference clone p0018.chsth71r (SEQ ID NOs: 1 and 2) and the genotype, tissue, and tissue treatment used for their isolation. The ubiquitin promoter may be used in an expression cassette.

TABLE 2

| | | | Amino Acid | |
|---|---|---|---|---|
| SEQ | | | Identity/ | |
| ID NO: | Database ID NO: | Species | Similarity | Isolation |
| 1, 2 | p0018.chsth71r | maize | 100/100 | B73 seedling, |
| | | | | V5-V7 stage |
| | | | | after 10 days of |
| | | | | drought stress |
| 3 | p0032.crcbg26r | maize | 89/92 | Hi-II callus |
| 4 | p0085.cscan24r | maize | 100/100 | Hi-II callus |
| | | | (over a short | |
| | | | region) | |
| 5 | p0095.cwsbh58r | maize | 55/76 | B73, ear leaf |
| | | | | sheath, 14-days |
| | | | | post pollination |
| 6 | p0126.cnleh06r | maize | 55/61 | B75, leaves, V8 |
| | | | | V10 stage |
| 7 | rds1f.pk002.a8 | rice | 84/89 | M103, |
| | | | | developing seed |
| 8 | rls24.pk0021.d7 | rice | 55/63 | Yashiro mochi, |
| | | | | 15-day old |
| | | | | plants, leaves, |
| | | | | infected with |
| | | | | fungus |
| | | | | Magnaporthe |
| | | | | grisea |
| 9 | wre1n.pk0130.d1 | wheat | 74/84 | Common, 7-day |
| | | | | old seedling |
| | | | | roots |

Agrobacterium mediated transformation and particle bombardment may be used for the introduction of DNA into host cells.

Definitions

Units, prefixes, and symbols may be denoted in their SI accepted form. Unless otherwise indicated, nucleic acids are written left to right in 5′ to 3′ orientation; amino acid sequence are written left to right in amino to carboxy orientation, respectively. Numeric ranges recited within the specification are inclusive of the numbers defining the range and include each integer within the defined range. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole. Section headings provided throughout the specification are not limitations to the various embodiments of the present invention.

By “amplified” the construction of multiple copies of a nucleic acid sequence or multiple copies complementary to the nucleic acid sequence using at least one of the nucleic acid sequences as a template is meant. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

As used herein, “antisense orientation” includes reference to a duplex polynucleotide sequence that is operably linked to a promoter in an orientation where the antisense strand is transcribed. The antisense strand is sufficiently complementary to an endogenous transcription product such that translation of the endogenous transcription product is often inhibited. Antisense constructions having at least about 70%, preferably 80%, and more preferably at least about 85% sequence identity to the antisense sequences of the invention may be used.

By “encoding” or “encoded”, comprising the information for translation into the specified protein with respect to a specified nucleic acid is meant. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al. (1989) Nucl Acids Res 17: 477-498). Thus, the maize preferred codon for a particular amino acid may be derived from known gene sequences from maize. Maize codon usage for 28 genes from maize plants is listed in Table 4 of Murray et al., Id.

As used herein “full-length sequence” in reference to a specified polynucleotide or its encoded protein means having the entire amino acid sequence of, a native (non-synthetic), endogenous, biologically (e.g., structurally or catalytically) active form of the specified protein. Methods to determine whether a sequence is full-length are well known in the art including such exemplary techniques as northern or western blots, primer extension, S1 protection, and ribonuclease protection. See, e.g., Plant Molecular Biology: A Laboratory Manual, Clark, Ed., Springer-Verlag, Berlin (1997). Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

As used herein, “heterologous”, in reference to a nucleic acid, is a nucleic acid that originates from a foreign species, or, if from the same species, is substantially modified from its native form in composition and/or genomic locus by human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by human intervention.

By “host cell” a cell that contains a vector and supports the replication and/or expression of the vector is meant. Host cells may be prokaryotic cells such as E. coli, or eukaryotic cells such as yeast, insect, amphibian, or mammalian cells. Preferably, host cells are monocotyledonous or dicotyledonous plant cells. A particularly preferred monocotyledonous host cell is a maize host cell.

The term “introduced” includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA). The term includes such nucleic acid introduction means as “transfection”, “transformation” and “transduction”.

The term “isolated” refers to material, such as a nucleic acid or a protein, which is substantially free from components that normally accompany or interact with it as found in its naturally occurring environment. The isolated material optionally comprises material not found with the material in its natural environment, or if the material is in its natural environment, the material has been synthetically (non-naturally) altered by human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein, are also referred to as “heterologous” nucleic acids.

As used herein, “nucleic acid” includes reference to a deoxyribonucleotide or ribonucleotide polymer, or chimeras thereof, in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” a collection of isolated DNA or RNA molecules which comprise and substantially represent the entire transcribed fraction of a genome of a specified organism, tissue, or of a cell type from that organism is meant. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (1987); Sambrook et al., Molecular Cloning—A Laboratory Manual, 2nd ed., Vol. 1-3 (1989); and Current Protocols in Molecular Biology, F. M. Ausubel et al, Eds., Current Protocols, a joint venture between Greene Publishing Associates, Inc. and John Wiley & Sons, Inc. (1994).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen, and microspores. The classes of plants which can be used in the methods of the invention include both monocotyledonous and dicotyledonous plants. A particularly preferred plant is Zea mays.

As used herein, “polynucleotide” includes reference to a deoxyribopolynucleotide, ribopolynucleotide, or chimeras or analogs thereof that have the essential nature of a natural deoxy- or ribo-nucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among others, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such as Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissue are referred to as “tissue specific”. A “cell type specific” promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of human intervention. The term “recombinant” as used herein does not encompass the alteration of the cell or vector by naturally occurring events (e.g., spontaneous mutation, natural transformation/transduction/transposition) such as those occurring without human intervention.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The terms “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, preferably 90% sequence identity, and most preferably 100% sequence identity (i.e., complementary) with each other.

The term “stringent conditions” or “stringent hybridization conditions” includes reference to conditions under which a probe will selectively hybridize to its target sequence, to a detectably greater degree than to other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1×to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5×to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. The Tm (thermal melting point) is the temperature (under defined ionic strength and pH) at which 50% of a complementary target sequence hybridizes to a perfectly matched probe. For DNA-DNA hybrids, the Tₘ can be approximated from the equation of Meinkoth and Wahl, Anal Biochem, 138: 267-284 (1984): Tₘ=81.5° C.+16.6 (log M)+0.41 (%GC)−0.61 (% form)−500/L; where M is the molarity of monovalent cations, %GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ).

Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, supra. The duration of hybridization is generally less than about 24 hours, usually from about 4 to about 12 hours.

As used herein, “transgenic plant” includes reference to a plant which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of a heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in introduction of a polynucleotide of the present invention into a host cell. Vectors are often replicons. Expression vectors permit transcription of a nucleic acid inserted therein.

The nucleotide and polypeptide sequences of the invention include those set forth in the sequence listing as well as sequences having at least about 65%, about 70%, about 80%, about 85%, about 90%, 91%, 92%, 93%, 94%, 95%, 96%, 97%, 98%, 99%, and including 100% sequence identity to the disclosed sequences. The following terms are used to describe the sequence relationships between a polynucleotide/polypeptide of the present invention with a reference polynucleotide/polypeptide: (a) “reference sequence”, (b) “comparison window”, (c) “sequence identity”, and (d) “percentage of sequence identity”.

(a) As used herein, “reference sequence” is a defined sequence used as a basis for sequence comparison with a polynucleotide/polypeptide of the present invention. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide/polypeptide sequence, wherein the polynucleotide/polypeptide sequence may be compared to a reference sequence and wherein the portion of the polynucleotide/polypeptide sequence in the comparison window may comprise additions or deletions (i.e., gaps) compared to the reference sequence (which does not comprise additions or deletions) for optimal alignment of the two sequences. Generally, the comparison window is at least 20 contiguous nucleotide/amino acid residues in length, and optionally can be 30, 40, 50, 100, or longer. Those of skill in the art understand that to avoid a high similarity to a reference sequence due to inclusion of gaps in the polynucleotide/polypeptide sequence, a gap penalty is typically introduced and is subtracted from the number of matches.

Methods of alignment of sequences for comparison are well known in the art. Optimal alignment of sequences for comparison may be conducted by the local homology algorithm of Smith and Waterman, Adv Appl Math 2: 482 (1981); by the homology alignment algorithm of Needleman and Wunsch, J Mol Biol 48: 443 (1970); by the search for similarity method of Pearson and Lipman, Proc Natl Acad Sci 85: 2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA. The CLUSTAL program is well described by Higgins and Sharp, Gene 73: 237-244 (1988); Higgins and Sharp, CABIOS 5: 151-153 (1989); Corpet, et al., Nucleic Acids Res 16: 10881-90 (1988); Huang, et al., Computer Applications in the Biosciences 8: 155-65 (1992), and Pearson, et al., Methods in Molecular Biology 24: 307-331 (1994).

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, supra.

Unless otherwise stated, sequence identity/similarity values provided herein refer to the value obtained using the BLAST 2.0 suite of programs using default parameters. Altschul et al., J Mol Biol, 215: 403-410 (1990); Altschul et al., Nucleic Acids Res. 25: 3389-3402 (1997).

Software for performing BLAST analyses is publicly available, e.g., through the National Center for Biotechnology Information (www.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for riucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc Natl Acad Sci USA 89: 10915 (1989)).

In addition to calculating percent sequence identity, the BLAST algorithm also performs a statistical analysis of the similarity between two sequences (see, e.g., Karlin & Altschul, Proc Natl Acad Sci USA 90: 5873-5877 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput Chem, 17: 149-163 (1993)) and XNU (Claverie and States, Comput Chem, 17: 191-201 (1993)) low-complexity filters can be employed alone or in combination.

GAP can also be used to compare a polynucleotide or polypeptide of the present invention with a reference sequence. GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 100. Thus, for example, the gap creation and gap extension penalties can each independently be: 0, 1, 2, 3, 4, 5, 6, 7, 8, 9, 10, 15, 20, 30, 40, 50, 60 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the Quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff & Henikoff, supra).

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic Biol Sci, 4: 11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The present invention provides, among other things, compositions and methods for modulating (i.e., increasing or decreasing) the level of polynucleotides and polypeptides of the present invention in plants to modulate plant pathogen resistance. In particular, the polynucleotides and polypeptides of the present invention can be expressed temporally or spatially, e.g., at developmental stages, in tissues, and/or in quantities, which are uncharacteristic of non-recombinantly engineered plants.

The present invention also provides isolated nucleic acids comprising polynucleotides of sufficient length and complementarity to a polynucleotide of the present invention to use as probes or amplification primers in the detection, quantitation, or isolation of gene transcripts. For example, isolated nucleic acids of the present invention can be used as probes in detecting deficiencies in the level of mRNA in screenings for desired transgenic plants, for detecting mutations in the gene (e.g., substitutions, deletions, or additions), for monitoring upregulation of expression or changes in enzyme activity in screening assays of compounds, for detection of any number of allelic variants (polymorphisms), orthologs, or paralogs of the gene, or for site directed mutagenesis in eukaryotic cells (see, e.g., U.S. Pat. No. 5,565,350). The isolated nucleic acids of the present invention can also be used for recombinant expression of their encoded polypeptides, or for use as immunogens in the preparation and/or screening of antibodies. The isolated nucleic acids of the present invention can also be employed for use in sense or antisense suppression of one or more genes of the present invention in a host cell, tissue, or plant. Attachment of chemical agents which bind, intercalate, cleave and/or crosslink to the isolated nucleic acids of the present invention can also be used to modulate transcription or translation.

The present invention also provides isolated proteins comprising a polypeptide of the present invention (e.g., preproenzyme, proenzyme, or enzymes). The present invention also provides proteins comprising at least one epitope from a polypeptide of the present invention. The proteins of the present invention can be employed in assays for enzyme agonists or antagonists of enzyme function, or for use as immunogens or antigens to obtain antibodies specifically immunoreactive with a protein of the present invention. Such antibodies can be used in assays for expression levels, for identifying and/or isolating nucleic acids of the present invention from expression libraries, for identification of homologous polypeptides from other species, or for purification of polypeptides of the present invention.

The present invention may be used for transformation of any plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants.

Nucleic Acids

The present invention provides, among other things, isolated nucleic acids of RNA, DNA, and analogs and/or chimeras thereof, comprising a polynucleotide of the present invention.

A polynucleotide of the present invention is inclusive of those in Table 1 and:

(a) an isolated polynucleotide encoding a polypeptide of the present invention such as those referenced in Table 1, including exemplary polynucleotides of the present invention;

(b) an isolated polynucleotide which is the product of amplification from a plant nucleic acid library using primer pairs which selectively hybridize under stringent conditions to loci within a polynucleotide of the present invention;

(c) an isolated polynucleotide which selectively hybridizes to a polynucleotide of (a) or (b);

(d) an isolated polynucleotide having a specified sequence identity with polynucleotides of (a), (b), or (c);

(e) an isolated polynucleotide encoding a protein having a specified number of contiguous amino acids from a prototype polypeptide, wherein the protein is specifically recognized by antisera elicited by presentation of the protein and wherein the protein does not detectably immunoreact to antisera which has been fully immunosorbed with the protein;

(f) complementary sequences of polynucleotides of (a), (b), (c), (d), or (e); and

(g) an isolated polynucleotide comprising at least a specific number of contiguous nucleotides from a polynucleotide of (a), (b), (c), (d), (e), or (f);

(h) an isolated polynucleotide from a full-length enriched cDNA library having the physico-chemical property of selectively hybridizing to a polynucleotide of (a), (b), (c), (d), (e), (f), or (g);

(i) an isolated polynucleotide made by the process of: 1) providing a full-length enriched nucleic acid library, 2) selectively hybridizing the polynucleotide to a polynucleotide of (a), (b), (c), (d), (e), (f), (g), or (h), thereby isolating the polynucleotide from the nucleic acid library.

A. Polynucleotides Encoding A Polypeptide of the Present Invention

As indicated in (a), above, the present invention provides isolated nucleic acids comprising a polynucleotide of the present invention, wherein the polynucleotide encodes a polypeptide of the present invention. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Thus, each silent variation of a nucleic acid which encodes a polypeptide of the present invention is implicit in each described polypeptide sequence and is within the scope of the present invention. Accordingly, the present invention includes polynucleotides of the present invention and polynucleotides encoding a polypeptide of the present invention.

B. Polynucleotides Amplified from a Plant Nucleic Acid Library

As indicated in (b), above, the present invention provides an isolated nucleic acid comprising a polynucleotide of the present invention, wherein the polynucleotides are amplified, under nucleic acid amplification conditions, from a plant nucleic acid library. Nucleic acid amplification conditions for each of the variety of amplification methods are well known to those of ordinary skill in the art. The plant nucleic acid library can be constructed from a monocot such as a cereal crop. Exemplary cereals include corn, sorghum, alfalfa, canola, wheat, or rice. The plant nucleic acid library can also be constructed from a dicot such as soybean. Zea mays lines B73, PHRE1, A632, BMS-P2#10, W23, and Mo17 are known and publicly available. Other publicly known and available maize lines can be obtained from the Maize Genetics Cooperation (Urbana, Ill.). Wheat lines are available from the Wheat Genetics Resource Center (Manhattan, Kans.).

The nucleic acid library may be a cDNA library, a genomic library, or a library generally constructed from nuclear transcripts at any stage of intron processing. cDNA libraries can be normalized to increase the representation of relatively rare cDNAs. In optional embodiments, the cDNA library is constructed using an enriched full-length cDNA synthesis method. Examples of such methods include Oligo-Capping (Maruyama, K. and Sugano, S., Gene 138: 171-174 (1994)), Biotinylated CAP Trapper (Carninci, et al. Genomics 37: 327-336 (1996)), and CAP Retention Procedure (Edery, E. et al. Mol Cell Biol 15: 3363-3371 (1995)). Rapidly growing tissues or rapidly dividing cells are preferred for use as an mRNA source for construction of a cDNA library. Growth stages of corn are described in “How a Corn Plant Develops,” Special Report No. 48, Iowa State University of Science and Technology Cooperative Extension Service, Ames, Iowa, Reprinted February 1993.

A polynucleotide of this embodiment (or subsequences thereof) can be obtained, for example, by using amplification primers which are selectively hybridized and primer extended, under nucleic acid amplification conditions, to at least two sites within a polynucleotide of the present invention, or to two sites within the nucleic acid which flank and comprise a polynucleotide of the present invention, or to a site within a polynucleotide of the present invention and a site within the nucleic acid which comprises it. Methods for obtaining 5′ and/or 3′ ends of a vector insert are well known in the art. See, e.g., RACE (Rapid Amplification of Complementary Ends) as described in Frohman, M. A., in PCR Protocols: A Guide to Methods and Applications, M. A. Innis, et al., Eds., (Academic Press, Inc., San Diego), pp. 28-38 (1990)); see also, U.S. Pat. No. 5,470,722, and Current Protocols in Molecular Biology, Unit 15.6, supra; Frohman and Martin, (1989) Techniques 1: 165.

Optionally, the primers are complementary to a subsequence of the target nucleic acid which they amplify but may have a sequence identity ranging from about 85% to 99% relative to the polynucleotide sequence which they are designed to anneal to. As those skilled in the art will appreciate, the sites to which the primer pairs will selectively hybridize are chosen such that a single contiguous nucleic acid can be formed under the desired nucleic acid amplification conditions. The primer length in nucleotides is selected from the group of integers consisting of from at least 15 to 50. Thus, the primers can be at least 15, 18, 20, 25, 30, 40, or 50 nucleotides in length. Those of skill will recognize that a lengthened primer sequence can be employed to increase specificity of binding (i.e., annealing) to a target sequence. A non-annealing sequence at the 5′ end of a primer (a “tail”) can be added, for example, to introduce a cloning site at the terminal ends of the amplicon.

The amplification products can be translated using expression systems well known to those of skill in the art. The resulting translation products can be confirmed as polypeptides of the present invention by, for example, assaying for the appropriate catalytic activity (e.g., specific activity and/or substrate specificity), or verifying the presence of one or more linear epitopes which are specific to a polypeptide of the present invention. Methods for protein synthesis from PCR derived templates are known in the art and available commercially. See, e.g., Amersham Life Sciences, Inc., Catalog '97, p.354

C. Polynucleotides Which Selectively Hybridize to a Polynucleotide of (A) or (B)

As indicated in (c), above, the present invention provides isolated nucleic acids comprising polynucleotides of the present invention, wherein the polynucleotides selectively hybridize, under selective hybridization conditions, to a polynucleotide of sections (A) or (B) as discussed above. Thus, the polynucleotides of this embodiment can be used for isolating, detecting, and/or quantifying nucleic acids comprising the polynucleotides of (A) or (B). For example, polynucleotides of the present invention can be used to identify, isolate, or amplify partial or full-length clones in a deposited library. In some embodiments, the polynucleotides are genomic or cDNA sequences isolated or otherwise complementary to a cDNA from a dicot or monocot nucleic acid library. Exemplary species of monocots and dicots include, but are not limited to: maize, canola, soybean, cotton, wheat, sorghum, sunflower, alfalfa, oats, sugar cane, millet, barley, and rice. The cDNA library comprises at least 50% to 95% full-length sequences (for example, at least 50%, 60%, 70%, 80%, 90%, or 95% full-length sequences). The cDNA libraries can be normalized to increase the representation of rare sequences. See, e.g., U.S. Pat. No. 5,482,845. Low stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% to 80% sequence identity and can be employed to identify orthologous or paralogous sequences.

D. Polynucleotides Having a Specific Sequence Identity with the Polynucleotides of (A), (B), or (C)

As indicated in (d), above, the present invention provides isolated nucleic acids comprising polynucleotides of the present invention, wherein the polynucleotides have a specified identity at the nucleotide level to a polynucleotide as disclosed above in sections (A), (B), or (C). Identity can be calculated using, for example, the BLAST or GAP algorithms under default conditions. The percentage of identity to a: reference sequence is at least 60% and, rounded upwards to the nearest integer, can be expressed as an integer selected from the group of integers consisting of from 60 to 99. Thus, for example, the percentage of identity to a reference sequence can be at least 70%, 75%, 80%, 85%, 90%, or 95%.

Optionally, the polynucleotides of this embodiment will encode a polypeptide that will share an epitope with a polypeptide encoded by the polynucleotides of sections (A), (B), or (C). Thus, these polynucleotides encode a first polypeptide which elicits production of antisera comprising antibodies which are specifically reactive to a second polypeptide encoded by a polynucleotide of (A), (B), or (C). However, the first polypeptide does not bind to antisera raised against itself when the antisera has been fully immunosorbed with the first polypeptide. Hence, the polynucleotides of this embodiment can be used to generate antibodies for use in, for example, the screening of expression libraries for nucleic acids comprising polynucleotides of (A), (B), or (C), or for purification of, or in imrniunoassays for, polypeptides encoded by the polynucleotides of (A), (B), or (C). The polynucleotides of this embodiment comprise nucleic acid sequences which can be employed for selective hybridization to a polynucleotide encoding a polypeptide of the present invention.

Screening polypeptides for specific binding to antisera can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 15 amino acids long. In addition to direct chemical synthetic methods for generating peptide libraries, several recombinant DNA methods have been described in the art. One type involves the display of a peptide sequence on the surface of a bacteriophage or cell. Each bacteriophage or cell contains the nucleotide sequence encoding the particular displayed peptide sequence. Such methods are described in WO 91/17271, 91/18980, 91/19818, and 93/08278. Other systems for generating libraries of peptides have aspects of both in vitro chemical synthesis and recombinant methods. See, WO 92/05258, 92/14843, and 97/20078. See also, U.S. Pat. Nos. 5,658,754; and 5,643,768. Peptide display libraries, vectors, and screening kits are commercially available from such suppliers as Invitrogen (Carlsbad, Calif.).

E. Polynucleotides Encoding a Protein Having a Subsequence from a Prototype Polypeptide and Cross-Reactive to the Prototype Polypeptide

As indicated in (e), above, the present invention provides isolated nucleic acids comprising polynucleotides of the present invention, wherein the polynucleotides encode a protein having a subsequence of contiguous amino acids from a prototype polypeptide of the present invention such as are provided in (a), above. The length of contiguous amino acids from the prototype polypeptide is selected from the group of integers consisting of from at least 10 to the number of amino acids within the prototype sequence. Thus, for example, the polynucleotide can encode a polypeptide having a subsequence having at least 10, 15, 20, 25, 30, 35, 40, 45, or 50, contiguous amino acids from the prototype polypeptide. Further, the number of such subsequences encoded by a polynucleotide of the instant embodiment can be any integer selected from the group consisting of from 1 to 20, such as 2, 3, 4, or 5. The subsequences can be separated by any integer of nucleotides from 1 to the number of nucleotides in the sequence such as at least 5, 10, 15, 25, 50, 100, or 200 nucleotides.

The proteins encoded by polynucleotides of this embodiment, when presented as an immunogen, elicit the production of polyclonal antibodies which specifically bind to a prototype polypeptide such as, but not limited to, a polypeptide encoded by the polynucleotide of (a) or (b), above. Generally, however, a protein encoded by a polynucleotide of this embodiment does not bind to antisera raised against the prototype polypeptide when the antisera has been fully immunosorbed with the prototype polypeptide. Methods of making and assaying for antibody binding specificity/affinity are well known in the art. Exemplary immunoassay formats include ELISA, competitive immunoassays, radioimmunoassays, Western blots, indirect immunofluorescent assays and the like.

In a preferred assay method, fully immunosorbed and pooled antisera which is elicited to the prototype polypeptide can be used in a competitive binding assay to test the protein. The concentration of the prototype polypeptide required to inhibit 50% of the binding of the antisera to the prototype polypeptide is determined. If the amount of the protein required to inhibit binding is less than twice the amount of the prototype protein, then the protein is said to specifically bind to the antisera elicited to the immunogen. Accordingly, the proteins of the present invention embrace allelic variants, conservatively modified variants, and minor recombinant modifications to a prototype polypeptide.

A polynucleotide of the present invention optionally encodes a protein having a molecular weight as the non-glycosylated protein within 20% of the molecular weight of the full-length non-glycosylated polypeptides of the present invention. Molecular weight can be readily determined by SDS-PAGE under reducing conditions. Optionally, the molecular weight is within 15% of a full-length polypeptide of the present invention, more preferably within 10% or 5%, and most preferably within 3%, 2%, or 1% of a full-length polypeptide of the present invention.

Optionally, the polynucleotides of this embodiment will encode a protein having a specific enzymatic activity of at least 50%, 60%, 70%, 80%, or 90% of a cellular extract comprising the native, endogenous full-length polypeptide of the present invention. Further, the proteins encoded by polynucleotides of this embodiment will optionally have a substantially similar affinity constant (Kₘ) and/or catalytic activity (i.e., the microscopic rate constant, k_{cat}) as the native endogenous, full-length protein. Those of skill in the art will recognize that the k_{cat}/Kₘ value determines the specificity for competing substrates and is often referred to as the specificity constant. Proteins of this embodiment can have a k_{cat}/Kₘ value at least 10% of a full-length polypeptide of the present invention as determined using the endogenous substrate of that polypeptide. Optionally, the k_{cat}/Kₘ value will be at least 20%, 30%, 40%, 50%, and most preferably at least 60%, 70%, 80%, 90%, or 95% the k_{cat}/Kₘ value of the full-length polypeptide of the present invention. Determination of k_{cat}, Kₘ, and k_{cat}/Kₘ can be determined by any number of means well known to those of skill in the art. For example, the initial rates (i.e., the first 5% or less of the reaction) can be determined using rapid mixing and sampling techniques (e.g., continuous-flow, stopped-flow, or rapid quenching techniques), flash photolysis, or relaxation methods (e.g., temperature jumps) in conjunction with such exemplary methods of measuring as spectrophotometry, spectrofluorimetry, nuclear magnetic resonance, or radioactive procedures. Kinetic values are conveniently obtained using a Lineweaver-Burk or Eadie-Hofstee plot.

F. Polynucleotides Complementary to the Polynucleotides of (A)-(E)

As indicated in (f), above, the present invention provides isolated nucleic acids comprising polynucleotides complementary to the polynucleotides of paragraphs A-E, above. As those of skill in the art will recognize, complementary sequences base-pair throughout the entirety of their length with the polynucleotides of sections (A)-(E) (i.e., have 100% sequence identity over their entire length). Complementary bases associate through hydrogen bonding in double stranded nucleic acids. For example, the following base pairs are complementary: guanine and cytosine; adenine and thymine; and adenine and uracil.

G. Polynucleotides Which are Subsequences of the Polynucleotides of (A)-(F)

As indicated in (g), above, the present invention provides isolated nucleic acids comprising polynucleotides which comprise at least 15 contiguous bases from the polynucleotides of sections (A) through (F) as discussed above. The length of the polynucleotide is given as an integer selected from the group consisting of from at least 15 to the length of the nucleic acid sequence from which the polynucleotide is a subsequence of. Thus, for example, polynucleotides of the present invention are inclusive of polynucleotides comprising at least 15, 20, 25, 30, 40, 50, 60, 75, or 100 contiguous nucleotides in length from the polynucleotides of (A)-(F). Optionally, the number of such subsequences encoded by a polynucleotide of the instant embodiment can be any integer selected from the group consisting of from 1 to 20, such as 2, 3, 4, or 5. The subsequences can be separated by any integer of nucleotides from 1 to the number of nucleotides in the sequence such as at least 5, 10, 15, 25, 50, 100, or 200 nucleotides.

Subsequences can be made by in vitro synthetic, in vitro biosynthetic, or in vivo recombinant methods. In optional embodiments, subsequences can be made by nucleic acid amplification. For example, nucleic acid primers will be constructed to selectively hybridize to a sequence (or its complement) within, or co-extensive with, the coding region.

The subsequences of the present invention can comprise structural characteristics of the sequence from which it is derived. Alternatively, the subsequences can lack certain structural characteristics of the larger sequence from which it is derived such as a poly (A) tail. Optionally, a subsequence from a polynucleotide encoding a polypeptide having at least one linear epitope in common with a prototype polypeptide sequence as provided in (a), above, may encode an epitope in common with the prototype sequence. Alternatively, the subsequence may not encode an epitope in common with the prototype sequence but can be used to isolate the larger sequence by, for example, nucleic acid hybridization with the sequence from which it is derived. Subsequences can be used to modulate or detect gene expression by introducing into the subsequences compounds which bind, intercalate, cleave and/or crosslink to nucleic acids. Exemplary compounds include acridine, psoralen, phenanthroline, naphthoquinone, daunomycin or chloroethylaminoaryl conjugates.

H. Polynucleotides From a Full-length Enriched cDNA Library Having the Physico-Chemical Property of Selectively Hybridizing to a Polynucleotide of (A)-(G)

As indicated in (h), above, the present invention provides an isolated polynucleotide from a full-length enriched cDNA library having the physico-chemical property of selectively hybridizing to a polynucleotide of paragraphs (A), (B), (C), (D), (E), (F), or (G) as discussed above. Methods of constructing full-length enriched cDNA libraries are known in the art. The cDNA library comprises at least 50% to 95% full-length sequences (for example, at least 50%, 60%, 70%, 80%, 90%, or 95% full-length sequences). The cDNA library can be constructed from a variety of tissues from a monocot or dicot at a variety of developmental stages. Exemplary species include maize, wheat, rice, canola, soybean, cotton, sorghum, sunflower, alfalfa, oats, sugar cane, millet, and barley. Methods of selectively hybridizing, under selective hybridization conditions, a polynucleotide from a full-length enriched library to a polynucleotide of the present invention, are known to those of ordinary skill in the art. Any number of stringency conditions can be employed to allow for selective hybridization. In optional embodiments, the stringency allows for selective hybridization of sequences having at least 70%, 75%, 80%, 85%, 90%, 95%, or 98% sequence identity over the length of the hybridized region. Full-length enriched cDNA libraries can be normalized to increase the representation of rare sequences.

I. Polynucleotide Products Made by an cDNA Isolation Process

As indicated in (i), above, the present invention provides an isolated polynucleotide made by the process of: 1) providing a full-length enriched nucleic acid library, 2) selectively hybridizing the polynucleotide to a polynucleotide of paragraphs (A), (B), (C), (D), (E), (F), (G, or (H) as discussed above, and thereby isolating the polynucleotide from the nucleic acid library. Full-length enriched nucleic acid libraries are constructed as discussed in paragraph (H) and below. Selective hybridization conditions are as discussed in paragraph (H). Nucleic acid purification procedures are well known in the art. Purification can be conveniently accomplished using solid-phase methods; such methods are well known to those of skill in the art and kits are available from commercial suppliers such as Advanced Biotechnologies (Surrey, UK). For example, a polynucleotide of paragraphs (A)-(H) can be immobilized to a solid support such as a membrane, bead, or particle. See, e.g., U.S. Pat. No. 5,667,976. The polynucleotide product of the present process is selectively hybridized to an immobilized polynucleotide and the solid support is subsequently isolated from non-hybridized polynucleotides by methods including, but not limited to, centrifugation, magnetic separation, filtration, electrophoresis, and the like.

Construction of Nucleic Acids

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a monocot. In preferred embodiments the monocot is Zea mays.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. A polynucleotide of the present invention can be attached to a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known and extensively described in the art. For a description of various nucleic acids see, for example, Stratagene Cloning Systems, Catalogs 1999 (La Jolla, Calif.); and, Amersham Life Sciences, Inc, Catalog '99 (Arlington Heights, Ill.).

A. Recombinant Methods for Constructing Nucleic Acids

The isolated nucleic acid compositions of this invention, such as RNA, cDNA, genomic DNA, or a hybrid thereof, can be obtained from plant biological sources using any number of cloning methodologies known to those of skill in the art. In some embodiments, oligonucleotide probes which selectively hybridize, under stringent conditions, to the polynucleotides of the present invention are used to identify the desired sequence in a cDNA or genomic DNA library. Isolation of RNA, and construction of cDNA and genomic libraries is well known to those of ordinary skill in the art. See, e.g., Plant Molecular Biology: A Laboratory Manual, supra; and, Current Protocols in Molecular Biology, supra.

A1. Full-length Enriched cDNA Libraries

A number of cDNA synthesis protocols have been described which provide enriched full-length cDNA libraries. Enriched full-length cDNA libraries are constructed to comprise at least 60%, and more preferably at least 70%, 80%, 90% or 95% full-length inserts amongst clones containing inserts. The length of insert in such libraries can be at least 2, 3, 4, 5, 6, 7, 8, 9, 10 or more kilobase pairs. Vectors to accommodate inserts of these sizes are known in the art and available commercially. See, e.g., Stratagene's lambda ZAP Express (cDNA cloning vector with 0 to 12 kb cloning capacity). An exemplary method of constructing a greater than 95% pure full-length cDNA library is described by Caminci et al, supra. Other methods for producing full-length libraries are known in the art. See, e.g., Edery et al., supra; and, PCT Application WO 96/34981.

A2. Normalized or Subtracted cDNA Libraries

A non-normalized cDNA library represents the mRNA population of the tissue it was made from. Since unique clones are out-numbered by clones derived from highly expressed genes their isolation can be laborious. Normalization of a cDNA library is the process of creating a library in which each clone is more equally represented. Construction of normalized libraries is described in Ko, Nucl Acids Res, 18(19): 5705-5711 (1990); Patanjali et al., Proc Natl Acad Sci USA, 88: 1943-1947 (1991); and U.S. Pat. Nos. 5,482,685, 5,482,845, and 5,637,685. In an exemplary method described by Soares et al., normalization resulted in reduction of the abundance of clones from a range of four orders of magnitude to a narrow range of only 1 order of magnitude. Proc Natl Acad Sci USA, 91: 9228-9232 (1994).

Subtracted cDNA libraries are another means to increase the proportion of less abundant cDNA species. In this procedure, cDNA prepared from one pool of mRNA is depleted of sequences present in a second pool of mRNA by hybridization. The cDNA:mRNA hybrids are removed and the remaining un-hybridized cDNA pool is enriched for sequences unique to that pool. See, Foote et al. in, Plant Molecular Biology: A Laboratory Manual, supra; Kho and Zarbl, Techniques, 3(2): 58-63 (1991); Sive and St. John, Nucl Acids Res, 16(22): 10937 (1988); Current Protocols in Molecular Biology, supra; and, Swaroop et al., Nucl Acids Res, 19(8): 1954 (1991). cDNA subtraction kits are commercially available. See, e.g., PCR-Select (Clontech, Palo Alto, Calif.).

To construct genomic libraries, large segments of genomic DNA are generated by fragmentation, by using restriction endonucleases, and are ligated with vector DNA to form concatemers that can be packaged into the appropriate vector. Methodologies to accomplish these ends, and sequencing methods to verify the sequence of nucleic acids are well known in the art. Examples of appropriate molecular biological techniques and instructions sufficient to direct persons of skill through many construction, cloning, and screening methodologies are found in Molecular Cloning—A Laboratory Manual, 2^{nd} Ed., supra; Guide to Molecular Cloning Techniques, Methods in Enzymology, Vol. 152, supra; Current Protocols in Molecular Biology, supra; Plant Molecular Biology: A Laboratory Manual, supra. Kits for construction of genomic libraries are also commercially available from a number of sources.

The cDNA or genomic library can be screened using a probe based upon the sequence of a polynucleotide of the present invention such as those disclosed herein. Probes may be used to hybridize with genomic DNA or cDNA sequences to isolate homologous genes in the same or different plant species. Those of skill in the art will appreciate that various degrees of stringency of hybridization can be employed in the assay; and either the hybridization or the wash medium can be stringent.

The nucleic acids of interest can also be amplified from nucleic acid samples using amplification techniques. For instance, PCR technology can be used to amplify the sequences of polynucleotides of the present invention and related genes directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. The T4 gene 32 protein (Boehringer Mannheim) can be used to improve yield of long PCR products.

PCR-based screening methods have been described. Wilfinger et al. describe a PCR-based method in which the longest cDNA is identified in the first step so that incomplete clones can be eliminated from study. BioTechniques, 22(3): 481-486 (1997). Such methods are particularly effective in combination with a full-length cDNA construction methodology described above.

B. Synthetic Methods for Constructing Nucleic Acids

The isolated nucleic acids of the present invention can also be prepared by direct chemical synthesis by methods such as the phosphotriester method of Narang et al., Meth Enzymol 68: 90-99 (1979); the phosphodiester method of Brown et al., Meth Enzymol 68: 109-151 (1979); the diethylphosphoramidite method of Beaucage and Caruthers, Tetra Lett 22: 1859-1862 (1981); the solid phase phosphoramidite triester method described by Beaucage and Caruthers, Id., e.g., using an automated synthesizer, e.g., as described in Needham-VanDevanter et al., Nucleic Acids Res, 12: 6159-6168 (1984); and, the solid support method of U.S. Pat. No. 4,458,066. Chemical synthesis generally produces a single stranded oligonucleotide. This may be converted into double stranded DNA by hybridization with a complementary sequence, or by polymerization with a DNA polymerase using the single strand as a template. One of skill will recognize that while chemical synthesis of DNA is best employed for sequences of about 100 bases or less, longer sequences may be obtained by the ligation of shorter sequences.

Recombinant Expression Cassettes

The present invention further provides recombinant expression cassettes comprising a nucleic acid of the present invention. A nucleic acid sequence coding for the desired polypeptide of the present invention, for example, a cDNA or a genomic sequence encoding a full-length polypeptide of the present invention, can be used to construct a recombinant expression cassette which can be introduced into the desired host cell. A recombinant expression cassette will typically comprise a polynucleotide of the present invention operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A plant promoter fragment can be employed which will direct expression of a polynucleotide of the present invention in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, and the GRP1-8 promoter.

Alternatively, the plant promoter can direct expression of a polynucleotide of the present invention in a specific tissue or may be otherwise under more precise environmental or developmental control. Such promoters are referred to here as “inducible” promoters. Environmental conditions that may effect transcription by inducible promoters include pathogen attack, anaerobic conditions, or the presence of light. Examples of inducible promoters are the Adh1 promoter which is inducible by hypoxia or cold stress, the Hsp70 promoter which is inducible by heat stress, and the PPDK promoter which is inducible by light.

Examples of promoters under developmental control include promoters that initiate transcription only, or preferentially, in certain tissues, such as leaves, roots, fruit, seeds, or flowers. Exemplary promoters include the anther specific promoter 5126 (U.S. Pat. Nos. 5,689,049 and 5,689,051), the ZRP2 promoter (U.S. Pat. No. 5,633,363), the IFS1 promoter (U.S. patent application Ser. No. 10/104,706), glob-1 promoter, and gamma-zein promoter. The operation of a promoter may also vary depending on its location in the genome. Thus, an inducible promoter may become fully or partially constitutive in certain locations.

Both heterologous and non-heterologous (i.e., endogenous) promoters can be employed to direct expression of the nucleic acids of the present invention. These promoters can also be used, for example, in recombinant expression cassettes to drive expression of antisense nucleic acids to reduce, increase, or alter the concentration and/or composition of the proteins of the present invention in a desired tissue. Thus, in some embodiments, the nucleic acid construct will comprise a promoter functional in a plant cell, such as in Zea mays, operably linked to a polynucleotide of the present invention. Promoters useful in these embodiments include the endogenous promoters driving expression of a polypeptide of the present invention.

In some embodiments, isolated nucleic acids which serve as promoter or enhancer elements can be introduced in the appropriate position (generally upstream) of a non-heterologous form of a polynucleotide of the present invention so as to up or down regulate expression of a polynucleotide of the present invention. For example, endogenous promoters can be altered in vivo by mutation, deletion, and/or substitution (see, U.S. Pat. No. 5,565,350; and WO 93/22443), or isolated promoters can be introduced into a plant cell in the proper orientation and distance from a cognate gene of a polynucleotide of the present invention so as to control the expression of the gene. Gene expression can be modulated under conditions suitable for plant growth so as to alter the total concentration and/or alter the composition of the polypeptides of the present invention in a plant cell. Thus, the present invention provides compositions, and methods for making, heterologous promoters and/or enhancers operably linked to a native, endogenous (i.e., non-heterologous) form of a polynucleotide of the present invention.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added can be derived from, for example, the nopaline synthase or octopine synthase genes, or alternatively from another plant gene, or less preferably from any other eukaryotic gene.

An intron sequence can be added to the 5′ untranslated region or the coding sequence or the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchmnan and Berg, Mol Cell Biol 8: 4395-4405 (1988); Callis et al., Genes Dev. 1: 1183-1200 (1987). Such intron enhancement of gene expression is typically greatest when placed near the 5′ end of the transcription unit. Use of maize introns Adh1-S intron 1, 2, and 6, the Bronze-1 intron are known in the art. See generally, The Maize Handbook, Chapter 116, Freeling and Walbot, Eds., Springer, N.Y. (1994). The vector comprising the sequences from a polynucleotide of the present invention will typically comprise a marker gene which confers a selectable phenotype on plant cells. Typical vectors useful for expression of genes in higher plants are well known in the art and include vectors derived from the tumor-inducing (Ti) plasmid of Agrobacterium tumefaciens described by Rogers et al., Meth in Enzymol, 153: 253-277 (1987).

A polynucleotide of the present invention can be expressed in either sense or anti-sense orientation as desired. It will be appreciated that control of gene expression in either sense or anti-sense orientation can have a direct impact on the observable plant characteristics. Antisense technology can be conveniently used to inhibit gene expression in plants. To accomplish this, a nucleic acid segment from the desired gene is cloned and operably linked to a promoter such that the anti-sense strand of RNA will be transcribed. The construct is then transformed into plants and the antisense strand of RNA is produced. In plant cells, it has been shown that antisense RNA inhibits gene expression by preventing the accumulation of mRNA which encodes the enzyme of interest, see, e.g., Sheehy et al., Proc Natl Acad Sci USA 85: 8805-8809 (1988); and U.S. Pat. No. 4,801,540.

Another method of suppression is sense suppression (i.e., co-suppression). Introduction of a nucleic acid configured in the sense orientation has been shown to be an effective means by which to block the transcription of target genes. For an example of the use of this method to modulate expression of endogenous genes see, Napoli et al., The Plant Cell 2: 279-289 (1990) and U.S. Pat. No. 5,034,323.

Catalytic RNA molecules or ribozymes can also be used to inhibit expression of plant genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloff et al., Nature 334: 585-591 (1988).

A variety of cross-linking agents, alkylating agents and radical generating species as pendant groups on polynucleotides of the present invention can be used to bind, label, detect, and/or cleave nucleic acids. For example, Vlassov, V. V., et al., Nucleic Acids Res (1986) 14: 4065-4076, describe covalent bonding of a single-stranded DNA fragment with alkylating derivatives of nucleotides complementary to target sequences. A report of similar work by the same group is that by Knorre, D. G., et al., Biochimie (1985) 67: 785-789. Iverson and Dervan also showed sequence-specific cleavage of single-stranded DNA mediated by incorporation of a modified nucleotide which was capable of activating cleavage (J Am Chem Soc (1987) 109: 1241-1243). Meyer, R. B., et al., J Am Chem Soc (1989) 111: 8517-8519, disclose covalent crosslinking to a target nucleotide using an alkylating agent complementary to the single-stranded target nucleotide sequence. A photoactivated crosslinking to single-stranded oligonucleotides mediated by psoralen was disclosed by Lee, B. L., et al., Biochemistry (1988) 27: 3197-3203. Use of crosslinking in triple-helix forming probes was also disclosed by Home, et al., J Am Chem Soc (1990) 112: 2435-2437. The use of N4, N4-ethanocytosine as an alkylating agent to crosslink to single-stranded oligonucleotides has also been described by Webb and Matteucci, J Am Chem Soc (1986) 108: 2764-2765; Nucleic Acids Res (1986) 14: 7661-7674; Feteritz et al., J Am Chem Soc 113: 4000 (1991). Various compounds to bind, detect, label, and/or cleave nucleic acids are known in the art. See, for example, U.S. Pat. Nos. 5,543,507; 5,672,593; 5,484,908; 5,256,648; and, 5,681941.

Proteins

The isolated proteins of the present invention comprise a polypeptide having at least 10 amino acids from a polypeptide of the present invention (or conservative variants thereof) such as those encoded by any one of the polynucleotides of the present invention as discussed more fully above (e.g., Table 1). The proteins of the present invention or variants thereof can comprise any number of contiguous amino acid residues from a polypeptide of the present invention, wherein that number is selected from the group of integers consisting of from 10 to the number of residues in a full-length polypeptide of the present invention. Optionally, this subsequence of contiguous amino acids is at least 15, 20, 25, 30, 35, or 40 amino acids in length, often at least 50, 60, 70, 80, or 90 amino acids in length. Further, the number of such subsequences can be any integer selected from the group consisting of from 1 to 20, such as 2, 3, 4, or 5.

The present invention further provides a protein comprising a polypeptide having a specified sequence identity with a polypeptide of the present invention. The percentage of sequence identity is an integer selected from the group consisting of from 50 to 99. Exemplary sequence identity values include 60%, 65%, 70%, 75%, 80%, 85%, 90%, and 95%. Sequence identity can be determined using, for example, the GAP or BLAST algorithms.

As those of skill will appreciate, the present invention includes, but is not limited to, catalytically active polypeptides of the present invention (i.e., enzymes). Catalytically active polypeptides have a specific activity of at least 20%, 30%, or 40%, and preferably at least 50%, 60%, or 70%, and most preferably at least 80%, 90%, or 95% that of the native (non-synthetic), endogenous polypeptide. Further, the substrate specificity (k_{cat}/Kₘ) is optionally substantially similar to the native (non-synthetic), endogenous polypeptide. Typically, the Kₘ will be at least 30%, 40%, or 50%, that of the native (non-synthetic), endogenous polypeptide; and more preferably at least 60%, 70%, 80%, or 90%. Methods of assaying and quantifying enzymatic activity and substrate specificity (k_{cat}/Kₘ), are well known to those of skill in the art.

Generally, the proteins of the present invention will, when presented as an immunogen, elicit production of an antibody specifically reactive to a polypeptide of the present invention. Further, the proteins of the present invention will not bind to antisera raised against a polypeptide of the present invention which has been fully immunosorbed with the same polypeptide. Immunoassays for determining binding are well known to those of skill in the art. A preferred immunoassay is a competitive immunoassay. Thus, the proteins of the present invention can be employed as immunogens for constructing antibodies immunoreactive to a protein of the present invention for such exemplary utilities as immunoassays or protein purification techniques.

Expression of Proteins in Host Cells

Using the nucleic acids of the present invention, one may express a protein of the present invention in a recombinantly engineered cell such as a bacteria, yeast, insect, mammalian, or preferably plant cell. The cells produce the protein in a non-natural condition (e.g., in quantity, composition, location, and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, however, the expression of isolated nucleic acids encoding a protein of the present invention will typically be achieved by operably linking, for example, the DNA or cDNA to a promoter (which is either constitutive or regulatable), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain a high level of expression of a cloned gene, it is desirable to construct expression vectors which contain, at a minimum, a strong promoter to direct transcription, a ribosome binding site for translational initiation, and a transcription/translation terminator. One of skill would recognize that modifications can be made to a protein of the present invention without diminishing its biological activity. Some modifications may be made to facilitate the cloning, expression, or incorporation of the targeting molecule. into a fusion protein. Such modifications are well known to those of skill in the art and include, for example, a methionine added at the amino terminus to provide an initiation site, or additional amino acids (e.g., poly His) placed on either terminus to create conveniently located purification sequences. Restriction sites or termination codons can also be introduced.

Synthesis of Proteins

The proteins of the present invention can be constructed using non-cellular synthetic methods. Solid phase synthesis of proteins of less than about 50 amino acids in length may be accomplished by attaching the C-terminal amino acid of the sequence to an insoluble support followed by sequential addition of the remaining amino acids in the sequence. Techniques for solid phase synthesis are described by Barany and Merrifield, Solid-Phase Peptide Synthesis, pp. 3-284 in The Peptides: Analysis, Synthesis, Biology. Vol. 2: Special Methods in Peptide Synthesis, Part A.; Merrifield, et al., J Am Chem Soc 85: 2149-2156 (1963), and Stewart et al., Solid Phase Peptide Synthesis, 2^{nd} Ed., Pierce Chem. Co., Rockford, Ill. (1984). Proteins of greater length may be synthesized by condensation of the amino and carboxy termini of shorter fragments. Methods of forming peptide bonds by activation of a carboxy terminal end (e.g., by the use of the coupling reagent N,N′-dicycylohexylcarbodiimide) are known to those of skill in the art.

Purification of Proteins

The proteins of the present invention may be purified by standard techniques well known to those of skill in the art. Recombinantly produced proteins of the present invention can be directly expressed or expressed as a fusion protein. The recombinant protein is purified by a combination of cell lysis (e.g., sonication, French press) and affinity chromatography. For fusion products, subsequent digestion of the fusion protein with an appropriate proteolytic enzyme releases the desired recombinant protein.

The proteins of this invention, recombinant or synthetic, may be purified to substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: New York (1982); Deutscher, Guide to Protein Purification, Academic Press (1990). For example, antibodies may be raised to the proteins as described herein. Purification from E. coli can be achieved following procedures described in U.S. Pat. No. 4,511,503. The protein may then be isolated from cells expressing the protein and further purified by standard protein chemistry techniques as described herein. Detection of the expressed protein is achieved by methods known in the art and include, for example, radioimmunoassays, Western blotting techniques or immunoprecipitation.

Introduction of Nucleic Acids Into Host Cells

The method of introducing a nucleic acid of the present invention into a host cell is not critical to the instant invention. Transformation or transfection methods are conveniently used. Accordingly, a wide variety of methods have been developed to insert a DNA sequence into the genome of a host cell to obtain the transcription and/or translation of the sequence to effect phenotypic changes in the organism. Thus, any method which provides for effective introduction of a nucleic acid may be employed.

A. Plant Transformation

A nucleic acid comprising a polynucleotide of the present invention is optionally introduced into a plant. Generally, the polynucleotide will first be incorporated into a recombinant expression cassette or vector. Isolated nucleic acids of the present invention can be introduced into plants according to techniques known in the art. Techniques for transforming a wide variety of higher plant species are well known and described in the technical, scientific, and patent literature. See, for example, Weising et al., Ann Rev Genet 22: 421-477 (1988). For example, the DNA construct may be introduced directly into the genomic DNA of the plant cell using techniques such as electroporation, polyethylene glycol (PEG), poration, particle bombardment, silicon fiber delivery, or microinjection of plant cell protoplasts or embryogenic callus. See, e.g., Tomes, et al., Direct DNA Transfer into Intact Plant Cells Via Microprojectile Bombardment. pp.197-213 in Plant Cell, Tissue and Organ Culture, Fundamental Methods. Eds., O. L. Gamborg and G. C. Phillips, Springer-Verlag Berlin, 1995; see, U.S. Pat. No. 5,990,387. The introduction of DNA constructs using PEG precipitation is described in Paszkowski et al., EMBO J 3: 2717-2722 (1984). Electroporation techniques are described in Fromm et al., Proc Natl Acad Sci USA 82: 5824 (1985). Ballistic transformation techniques are described in Klein et al., Nature 327: 70-73 (1987).

Agrobacterium tumefaciens-mediated transformation techniques are well described in the scientific literature. See, for example Horsch et al., Science 233: 496-498 (1984); Fraley et al., Proc Natl Acad Sci (USA) 80: 4803 (1983); U.S. Pat. No. 5,563,055; U.S. Pat. No. 5,981,840; and, Plant Molecular Biology: A Laboratory Manual, Chapter 8, supra. The DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. See, U.S. Pat. No. 5,591,616. Although Agrobacterium is useful primarily in dicots, certain monocots can be transformed by Agrobacterium. For instance, Agrobacterium transformation of maize is described in U.S. Pat. No. 5,550,318.

Other methods of transfection or transformation include (1) Agrobacterium rhizogenes-mediated transformation (see, e.g., Lichtenstein and Fuller In: Genetic Engineering, Vol. 6, PWJ Rigby, Ed., London, Academic Press, 1987; and Lichtenstein, C. P., and Draper, J,. In: DNA Cloning, Vol. II, D. M. Glover, Ed., Oxford, IRI Press, 1985); WO 88/02405 describes the use of A. rhizogenes strain A4 and its Ri plasmid along with A. tumefaciens vectors pARC8 or pARC16, (2) liposome-mediated DNA uptake (see, e.g., Freeman et al., Plant Cell Physiol 25: 1353 (1984)), and (3) the vortexing method (see, e.g., Kindle, Proc Natl Acad Sci USA 87: 1228 (1990)).

DNA can also be introduced into plants by direct DNA transfer into pollen as described by Zhou et al., Methods in Enzymology, 101: 433 (1983); D. Hess, Intern Rev Cytol, 107: 367 (1987); Luo et al., Plant Mol Biol Reporter, 6: 165 (1988). Expression of polypeptide coding genes can be obtained by injection of the DNA into reproductive organs of a plant as described by Pena et al., Nature, 325: 274 (1987). DNA can also be injected directly into the cells of immature embryos and the rehydration of desiccated embryos as described by Neuhaus et al., Theor Appl Genet, 75: 30 (1987); and Benbrook et al., in Proceedings Bio Expo 1986, Butterworth, Stoneham, Mass., pp. 27-54 (1986). A variety of plant viruses that can be employed as vectors are known in the art and include cauliflower mosaic virus (CaMV), geminivirus, brome mosaic virus, and tobacco mosaic virus.

B. Transfection of Prokaryotes, Lower Eukaryotes, and Animal Cells

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, electroporation, biolistics, and micro-injection of the DNA directly into the cells. The transfected cells are cultured by means well known in the art. See, Kuchler, R. J., Biochemical Methods in Cell Culture and Virology, Dowden, Hutchinson and Ross, Inc. (1977).

Transgenic Plant Regeneration

Plant cells which directly result or are derived from nucleic acid introduction techniques can be cultured to regenerate a whole plant which possesses the introduced genotype. Such regeneration techniques often rely on manipulation of certain phytohormones in a tissue culture growth medium. Plants cells can be regenerated, e.g., from single cells, callus tissue or leaf discs according to standard plant tissue culture techniques. It is well known in the art that various cells, tissues, and organs from almost any plant can be successfully cultured to regenerate an entire plant. Plant regeneration from cultured protoplasts is described in Evans et al., Protoplasts Isolation and Culture, Handbook of Plant Cell Culture, Macmillan Publishing Company, New York, pp. 124-176 (1983); and Binding, Regeneration of Plants, Plant Protoplasts, CRC Press, Boca Raton, pp. 21-73 (1985).

The regeneration of plants from either single plant protoplasts or various explants is well known in the art. See, for example, Methods for Plant Molecular Biology, A. Weissbach and H. Weissbach, Eds., Academic Press, Inc., San Diego, Calif. (1988). This regeneration and growth process includes the steps of selection of transformant cells and shoots, rooting the transformant shoots and growth of the plantlets in soil. For maize cell culture and regeneration see generally, The Maize Handbook, supra; Corn and Corn Improvement, 3^{rd} edition, Sprague and Dudley Eds., American Society of Agronomy, Madison, Wis. (1988). For transformation and regeneration of maize see, Tomes et al. “Direct DNA Transfer into Intact Plant Cells via Microprojectile Bombardment,” in Plant Cell, Tissue, and Organ Culture: Fundamental Methods, Eds., Gamborg and Phillips (Springer-Verlag, Berlin) (1995).

The regeneration of plants containing the polynucleotide of the present invention and introduced by Agrobacterium from leaf explants can be achieved as described by Horsch et al., Science, 227: 1229-1231 (1985). In this procedure, transformants are grown in the presence of a selection agent and in a medium that induces the regeneration of shoots in the plant species being transformed as described by Fraley et al., supra. This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

One of skill will recognize that after the recombinant expression cassette is stably incorporated in transgenic plants and confirmed to be operable, it can be introduced into other plants by sexual crossing. Any of a number of standard breeding techniques can be used, depending upon the species to be crossed. In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self-crossed to produce a homozygous inbred plant. The inbred plant produces seed containing the newly introduced heterologous nucleic acid. These seeds can be grown to produce plants that produce the selected phenotype. Parts obtained from the regenerated plant, such as flowers, seeds, leaves, branches, fruit, and the like are included in the invention, provided that these parts comprise cells comprising the isolated nucleic acid of the present invention. Progeny, variants, and mutants of the regenerated plants are also included within the scope of the invention, provided that these parts comprise the introduced nucleic acid sequences.

Transgenic plants expressing a polynucleotide of the present invention can be screened for transmission of the nucleic acid of the present invention by, for example, standard immunoblot and DNA detection techniques. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then be analyzed for protein expression by Western immunoblot analysis using the specifically reactive antibodies of the present invention. In addition, in situ hybridization and immunocytochemistry according to standard protocols can be done using heterologous nucleic acid specific polynucleotide probes and antibodies, respectively, to localize sites of expression within transgenic tissue. Generally, a number of transgenic lines are usually screened for the incorporated nucleic acid to identify and select plants with the most appropriate expression profiles.

A preferred embodiment is a transgenic plant that is homozygous for the added heterologous nucleic acid; i.e., a transgenic plant that contains two added nucleic acid sequences, one gene at the same locus on each chromosome of a chromosome pair. A homozygous transgenic plant can be obtained by sexually mating (selfing) a heterozygous transgenic plant that contains a single added heterologous nucleic acid, germinating some of the seed produced and analyzing the resulting plants produced for altered expression of a polynucleotide of the present invention relative to a control plant (i.e., native, non-transgenic). Back-crossing to a parental plant and out-crossing with a non-transgenic plant are also contemplated.

Modulating Polypeptide Levels and/or Composition

The present invention further provides a method for modulating (i.e., increasing or decreasing) the concentration or ratio of the polypeptides of the present invention in a plant or part thereof. Modulation can be effected by increasing or decreasing the concentration and/or the ratio of the polypeptides of the present invention in a plant. The method comprises introducing into a plant cell a recombinant expression cassette comprising a polynucleotide of the present invention as described above to obtain a transgenic plant cell, culturing the transgenic plant cell under transgenic plant cell growing conditions, and inducing or repressing expression of a polynucleotide of the present invention in the transgenic plant for a time sufficient to modulate the concentration and/or the ratios of the polypeptides in the transgenic plant or plant part.

In some embodiments, the concentration and/or ratios of polypeptides of the present invention in a plant may be modulated by altering, in vivo or in vitro, the promoter of a gene to up- or down-regulate gene expression. In some embodiments, the coding regions of native genes of the present invention can be altered via substitution, addition, insertion, or deletion to decrease activity of the encoded enzyme. See, U.S. Pat. 5,565,350; and WO 93/22443. And in some embodiments, an isolated nucleic acid (e.g., a vector) comprising a promoter sequence is transfected into a plant cell. Subsequently, a plant cell comprising the promoter operably linked to a polynucleotide of the present invention is selected for by means known to those of skill in the art such as, but not limited to, Southern blot, DNA sequencing, or PCR analysis using primers specific to the promoter and to the gene and detecting amplicons produced therefrom. A plant or plant part altered or modified by the foregoing embodiments is grown under plant forming conditions for a time sufficient to modulate the concentration and/or ratios of polypeptides of the present invention in the plant. Plant forming conditions are well known in the art.

In general, the concentration or the ratios of the polypeptides is increased or decreased by at least 5%, 10%, 20%, 30%, 40%, 50%, 60%, 70%, 80%, or 90% relative to a native control plant, plant part, or cell lacking the aforementioned recombinant expression cassette. Modulation in the present invention may occur during and/or subsequent to growth of the plant to the desired stage of development. Modulating nucleic acid expression temporally and/or in particular tissues can be controlled by employing the appropriate promoter operably linked to a polynucleotide of the present invention in, for example, sense or antisense orientation as discussed in greater detail, supra. Induction of expression of a polynucleotide of the present invention can also be controlled by exogenous administration of an effective amount of an inducing compound. Inducible promoters and inducing compounds which activate expression from these promoters are well known in the art. In one embodiment, the polypeptides of the present invention are modulated in monocots, particularly maize.

UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, Nucleic Acids Res 15: 8125 (1987)) and the 7-methylguanosine cap structure (Drummond et al., Nucleic Acids Res 13: 7375 (1985)). Negative elements include stable intramolecular 5′ UTR stem-loop structures (Muesing et al., Cell 48: 691 (1987)) and AUG sequences or short open reading frames preceded by an appropriate AUG in the 5′ UTR (Kozak, supra, and Rao et al., Mol Cell Biol. 8: 284 (1988)). Accordingly, the present invention provides 5′ and/or 3′ untranslated regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host such as to optimize the codon usage in a heterologous sequence for expression in maize. Codon usage in the coding regions of the polynucleotides of the present invention can be analyzed statistically using commercially available software packages such as “Codon Preference” available from the University of Wisconsin Genetics Computer Group (see Devereaux et al., Nucleic Acids Res 12: 387-395 (1984)) or MacVector 4.1 (Eastman Kodak Co., New Haven, Conn.). Thus, the present invention provides a codon usage frequency characteristic of the coding region of at least one of the polynucleotides of the present invention. The number of polynucleotides that can be used to determine a codon usage frequency can be any integer from 1 to the number of polynucleotides of the present invention as provided herein. Optionally, the polynucleotides will be full-length sequences. An exemplary number of sequences for statistical analysis can be at least 1, 5, 10, 20, 50, or 100.

Sequence Shuffling

The present invention provides methods for sequence shuffling using polynucleotides of the present invention, and compositions resulting therefrom. Sequence shuffling is described in WO 97/20078. See also, Zhang, J. H., et al. Proc Natl Acad Sci USA 94: 4504-4509 (1997). Generally, sequence shuffling provides a means for generating libraries of polynucleotides having a desired characteristic which can be selected or screened for. Libraries of recombinant polynucleotides are generated from a population of related sequence polynucleotides which comprise sequence regions which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include the properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property. In some embodiments, the selected characteristic will be a decreased Kₘ and/or increased K_{cat} over the wild-type protein as provided herein. In other embodiments, a protein or polynucleotide generated from sequence shuffling will have a ligand binding affinity greater than the non-shuffled wild-type polynucleotide. The increase in such properties can be at least 110%, 120%, 130%, 140% or at least 150% of the wild-type value.

Generic and Consensus Sequences

Polynucleotides and polypeptides of the present invention further include those having: (a) a generic sequence of at least two homologous polynucleotides or polypeptides, respectively, of the present invention; and, (b) a consensus sequence of at least three homologous polynucleotides or polypeptides, respectively, of the present invention. The generic sequence of the present invention comprises each species of polypeptide or polynucleotide embraced by the generic polypeptide or polynucleotide sequence, respectively. The individual species encompassed by a polynucleotide having an amino acid or nucleic acid consensus sequence can be used to generate antibodies or produce nucleic acid probes or primers to screen for homologs in other species, genera, families, orders, classes, phyla, or kingdoms. For example, a polynucleotide having a consensus sequence from a gene family of Zea mays can be used to generate antibody or nucleic acid probes or primers to other Gramineae species such as wheat, rice, or sorghum. Alternatively, a polynucleotide having a consensus sequence generated from orthologous genes can be used to identify or isolate orthologs of other taxa. Typically, a polynucleotide having a consensus sequence will be at least 9, 10, 15, 20, 25, 30, or 40 amino acids in length, or 20, 30, 40, 50, 100, or 150 nucleotides in length. As those of skill in the art are aware, a conservative amino acid substitution can be used for amino acids which differ amongst aligned sequences but are from the same conservative substitution group as discussed above. Optionally, no more than 1 or 2 conservative amino acids are substituted for each 10 amino acid length of consensus sequence.

Similar sequences used for generation of a consensus or generic sequence include any number and combination of allelic variants of the same gene, orthologous, or paralogous sequences as provided herein. Optionally, similar sequences used in generating a consensus or generic sequence are identified using the BLAST algorithm's smallest sum probability (P(N)). Various suppliers of sequence-analysis software are listed in Chapter 7 of Current Protocols in Molecular Biology, (Supplement 30), supra. A polynucleotide sequence is considered similar to a reference sequence if the smallest sum probability in a comparison of the test nucleic acid to the reference nucleic acid is less than about 0.1, more preferably less than about 0.01, or 0.001, and most preferably less than about 0.0001, or 0.00001. Similar polynucleotides can be aligned and a consensus or generic sequence generated using multiple sequence alignment software available from a number of commercial suppliers such as the Genetics Computer Group's (Madison, Wis.) PILEUP software, Vector NTI's (North Bethesda, Md.) ALIGNX, or Genecode's (Ann Arbor, Mich.) SEQUENCHER. Conveniently, default parameters of such software can be used to generate consensus or generic sequences.

Pathogens and Disease Resistance

The invention is drawn to compositions and methods for inducing resistance in a plant to plant pests. Accordingly, the compositions and methods are also useful in protecting plants against fungal pathogens, viruses, nematodes, insects and the like.

By “disease resistance” is intended that the plants avoid the disease symptoms that are the outcome of plant-pathogen interactions. That is, pathogens are prevented from causing plant diseases and the associated disease symptoms, or alternatively, the disease symptoms caused by the pathogen are minimized or lessened.

By “antipathogenic compositions” it is intended that the compositions of the invention have antipathogenic activity and thus are capable of suppressing, controlling, and/or killing the invading pathogenic organism. An antipathogenic composition of the invention will reduce the disease symptoms resulting from pathogen challenge by at least about 5% to about 50%, at least about 10% to about 60%, at least about 30% to about 70%, at least about 40% to about 80%, or at least about 50% to about 90% or greater. Hence, the methods of the invention can be utilized to protect plants from disease, particularly those diseases that are caused by plant pathogens.

Assays that measure antipathogenic activity are commonly known in the art, as are methods to quantitate disease resistance in plants following pathogen infection. See, for example, U.S. Pat. No. 5,614,395, herein incorporated by reference. Such techniques include, measuring over time, the average lesion diameter, the pathogen biomass, and the overall percentage of decayed plant tissues. For example, a plant either expressing an antipathogenic polypeptide or having an antipathogenic composition applied to its surface shows a decrease in tissue necrosis (i.e., lesion diameter) or a decrease in plant death following pathogen challenge when compared to a control plant that was not exposed to the antipathogenic composition. Alternatively, antipathogenic activity can be measured by a decrease in pathogen biomass. For example, a plant expressing an antipathogenic polypeptide or exposed to an antipathogenic composition is challenged with a pathogen of interest. Over time, tissue samples from the pathogen-inoculated tissues are obtained and RNA is extracted. The percent of a specific pathogen RNA transcript relative to the level of a plant specific transcript allows the level of pathogen biomass to be determined. See, for example, Thomma et al. (1998) Plant Biology 95: 15107-15111, herein incorporated by reference.

Furthermore, in vitro antipathogenic assays include, for example, the addition of varying concentrations of the antipathogenic composition to paper disks and placing the disks on agar containing a suspension of the pathogen of interest. Following incubation, clear inhibition zones develop around the discs that contain an effective concentration of the antipathogenic polypeptide (Liu et al. (1994) Plant Biology 91: 1888-1892, herein incorporated by reference). Additionally, microspectrophotometrical analysis can be used to measure the in vitro antipathogenic properties of a composition (Hu et al. (1997) Plant Mol Biol 34: 949-959 and Cammue et al. (1992) J Biol Chem 267: 2228-2233, both of which are herein incorporated by reference).

Pathogens of the invention include, but are not limited to, viruses or viroids, bacteria, insects, nematodes, fungi, and the like. Viruses include any plant virus, for example, tobacco or cucumber mosaic virus, ringspot virus, necrosis virus, maize dwarf mosaic virus, etc. Specific fungal and viral pathogens for the major crops include, but are not limited to: Soybeans: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines, Fusarium solani; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Alternaria alternata; Alfalfa: Clavibater michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium, Xanthomonas campestris p.v. alfalfae, Aphanomyces euteiches, Stemphylium herbarum, Stemphylium alfalfae; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Tilletia indica, Rhizoctonia solani, Pythium gramicola, High Plains Virus, European wheat striate virus; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Corn: Fusarium moniliforme var. subglutinans, Erwinia stewartii, Fusarium moniliforme, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H Virus, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola; Rice: Ceratobasidium oryzae-sativae, Curvularia lunata, Pyricularia grisea, Cochliobolus miyabeanus (Bipolaris oryzae), Gaeumannomyces gramini, Sclerophthora macrospora, Drechslera gigantea, Ustilaginoidea virens, Tilletia barclayana, Entyloma oryzae, Microdochium oryzae (Rhynchosporium oryzae), Cercospora janseana, Sarocladium oryzae, Fusarium spp., Pythium spp., Rhizoctonia solani, Sclerotium rolfsii, Thanatephorus cucumeris, Sarocladium oryzae, Rhizoctonia oryzae, Alternaria padwickii, Magnaporthe salvinii, Achlya conspicua, A. klebsiana, Rice Black-Streaked Dwarf Virus, Rice Bunchy Stunt Virus, Rice Dwarf Virus, Rice Gall Dwarf Virus, Rice Giallume Virus, Rice Grassy Stunt Virus, Rice Hoja Blanca Virus, Rice Necrosis Mosaic Virus, Rice Ragged Stunt Virus, Rice Stripe Necrosis Virus, Rice Stripe Virus, Rice Transitory Yellowing Virus, Rice Tungro Bacilliform Virus, Rice Tungro Spherical Virus, and Rice Yellow Mottle Virus.

Nematodes include parasitic nematodes such as root-knot, cyst, and lesion nematodes, including Heterodera and Globodera spp; particularly Globodera rostochiensis and globodera pailida (potato cyst nematodes); Heterodera glycines (soybean cyst nematode); Heterodera schachtii (beet cyst nematode); Heterodera avenae (cereal cyst nematode); Aphelenchoides besseyi (crimp nematode); Meloidogyne spp. (root knot nematode); Hirschmaniella oryzae (rice root nematode) and Ditylenchus angustus (rice stem nematode).

Detection of Nucleic Acids

The present invention further provides methods for detecting a polynucleotide of the present invention in a nucleic acid sample suspected of containing a polynucleotide of the present invention, such as a plant cell lysate, particularly a lysate of maize. In some embodiments, a cognate gene of a polynucleotide of the present invention or portion thereof can be amplified prior to the step of contacting the nucleic acid sample with a polynucleotide of the present invention. The nucleic acid sample is contacted with the polynucleotide to form a hybridization complex. The polynucleotide hybridizes under stringent conditions to a gene encoding a polypeptide of the present invention. Formation of the hybridization complex is used to detect a gene encoding a polypeptide of the present invention in the nucleic acid sample. Those of skill will appreciate that an isolated nucleic acid comprising a polynucleotide of the present invention should lack cross-hybridizing sequences in common with non-target genes that would yield a false positive result. Detection of the hybridization complex can be achieved using any number of well known methods. For example, the nucleic acid sample, or a portion thereof, may be assayed by hybridization formats, including but not limited to, solution phase, solid phase, mixed phase, or in situ hybridization assays.

Detectable labels suitable for use in the present invention include any composition detectable by spectroscopic, radioisotopic, photochemical, biochemical, immunochemical, electrical, optical or chemical means. Useful labels in the present invention include biotin for staining with labeled streptavidin conjugate, magnetic beads, fluorescent dyes, radiolabels, enzymes, and calorimetric labels. Other labels include ligands which bind to antibodies labeled with fluorophores, chemiluminescent agents, and enzymes. Labeling of the nucleic acids of the present invention is readily achieved by the use of labeled PCR primers and other methods known in the art.

Although the present invention has been described in some detail by way of illustration and example for purposes of clarity of understanding, it will be obvious that certain changes and modifications may be practiced within the scope of the appended claims. The following examples are offered by way of illustration and not by way of limitation.

### EXAMPLES

### Example 1

This example describes the construction of a cDNA library.

Total RNA can be isolated from maize tissues with TRIzol Reagent (Life Technology Inc. Gaithersburg, Md.) using a modification of the guanidine isothiocyanate/acid-phenol procedure described by Chomczynski and Sacchi (N Anal Biochem 162: 156 (1987)). In brief, a plant tissue sample is pulverized in liquid nitrogen before the addition of the TRIzol Reagent, and then further homogenized with a mortar and pestle. Addition of chloroform followed by centrifugation is conducted for separation of an aqueous phase and an organic phase. The total RNA is recovered by precipitation with isopropyl alcohol from the aqueous phase.

The selection of poly(A)+RNA from total RNA can be performed using the PolyATact system (Promega Corporation, Madison, Wis.). Biotinylated oligo(dT) primers are used to hybridize to the 3′ poly(A) tails on mRNA. The hybrids are captured using streptavidin coupled to paramagnetic particles and a magnetic separation stand. The mRNA is then washed at high stringency conditions and eluted by RNase-free deionized water.

cDNA synthesis and construction of unidirectional cDNA libraries can be accomplished using the SuperScript Plasmid System (Life Technology Inc. Gaithersburg, Md.). The first strand of cDNA is synthesized by priming an oligo(dT) primer containing a Not I site. The reaction is catalyzed by SuperScript Reverse Transcriptase II at 45° C. The second strand of cDNA is labeled with alpha-³²P-dCTP and a portion of the reaction analyzed by agarose gel electrophoresis to determine cDNA sizes. cDNA molecules smaller than 500 base pairs and unligated adapters are removed by Sephacryl-S400 chromatography. The selected cDNA molecules are ligated into pSPORT1 vector in between of Not I and Sal I sites.

Alternatively, cDNA libraries can be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts will be contained in the plasmid vector pBluescript. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBluescript plasmids, or the insert cDNA sequences are amplified via polymerase chain reaction (PCR) using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252: 1651-1656). The resulting ESTs are analyzed using a Perkin Elmer Model 377 fluorescent sequencer.

### Example 2

This example describes construction of a full-length enriched cDNA library.

An enriched full-length cDNA library can be constructed using one of two variations of the method of Carninci et al., supra. These variations are based on chemical introduction of a biotin group into the diol residue of the 5′ cap structure of eukaryotic mRNA to select full-length first strand cDNA. The selection occurs by trapping the biotin residue at the cap sites using streptavidin-coated magnetic beads followed by RNase I treatment to eliminate incompletely synthesized cDNAs. Second strand cDNA is synthesized using established procedures such as those provided in Life Technologies' (Rockville, Md.) “SuperScript Plasmid System for cDNA Synthesis and Plasmid Cloning” kit. Libraries made by this method have been shown to contain 50% to 70% full-length cDNAs.

The first strand synthesis methods are detailed below. An asterisk denotes that the reagent was obtained from Life Technologies, Inc.

A. First strand cDNA synthesis Method 1 (with trehalose)

| | | |
|---|---|---|
| mRNA (10 μg) | 25 | μl |
| *Not I primer (5 μg) | 10 | μl |
| *5x 1^{st} strand buffer | 43 | μl |
| *0.1m DTT | 20 | μl |
| *dNTP mix 10 mm | 10 | μl |
| BSA 10 μg/μl | 1 | μl |
| Trehalose (saturated) | 59.2 | μl |
| RNase inhibitor (Promega) | 1.8 | μl |
| *Superscript II RT 200 u/μl | 20 | μl |
| 100% glycerol | 18 | μl |
| Water | 7 | μl |

The mRNA and Not I primer are mixed and denatured at 65° C. for 10 min. They are then chilled on ice and other components added to the tube. Incubation is at 45° C. for 2 min. Twenty microliters of RT (reverse transcriptase) is added to the reaction and the reaction program is started on the thermocycler (MJ Research, Waltham, Mass.):

| | |
|---|---|
| Step 1 | 45° C. 10 min |
| Step 2 | 45° C. −0.3° C./cycle , 2 sec/cycle |
| Step 3 | Go to 2 for 33 cycles |
| Step 4 | 35° C. 5 min |
| Step 5 | 45° C. 5 min |
| Step 6 | 45° C. 0.2° C./cycle, 1 sec/cycle |
| Step 7 | Go to 6 for 49 cycles |
| Step 8 | 55° C. 0.1° C./cycle, 12 sec/cycle |
| Step 9 | Go to 8 for 49 cycles |
| Step 10 | 55° C. 2 min |
| Step 11 | 60° C. 2 min |
| Step 12 | Goto 11 for 9 times |
| Step 13 | 4° C. |
| Step 14 | End |

B. First strand cDNA synthesis Method 2

| | | |
|---|---|---|
| mRNA (10 μg) | 25 | μl |
| Water | 30 | μl |
| *Not I adapter primer (5 μg) | 10 | μl |
| 65° C. for 10 min, chill on ice, | | |
| then add following reagents, | | |
| *5x first buffer | 20 | μl |
| *0.1 M DTT | 10 | μl |
| *10 mM dNTP mix | 5 | μl |

Incubate at 45° C. for 2 min, then add 10 μl of *Superscript II RT (200 u/μl), start the following program:

| | |
|---|---|
| Step 1 | 45° C. for 6 sec, −0.1° C./cycle |
| Step 2 | Go to 1 for 99 additional cycles |
| Step 3 | 35° C. for 5 min |
| Step 4 | 45° C. for 60 min |
| Step 5 | 50° C. for 10 min |
| Step 6 | 4° C. |
| Step 7 | End |

After the 1st strand cDNA synthesis, the DNA is extracted by phenol according to standard procedures, and then precipitated in NaOAc and ethanol, and stored in −20° C.

C. Oxidization of the Diol Group of mRNA for Biotin Labeling

First strand cDNA is spun down and washed once with 70% EtOH. The pellet is resuspended in 23.2 μl of DEPC treated water and put on ice. 100 mM of NaIO₄ is freshly prepared, and then the following reagents are added:

| | | |
|---|---|---|
| mRNA:1^{st} cDNA (start with 20 μg mRNA) | 46.4 | μl |
| 100 mM NaIO4 (freshly made) | 2.5 | μl |
| NaOAc 3M pH 4.5 | 1.1 | μl |

To make 100 mM NaIO₄, use 21.391 μg of NaIO₄ for 1 μl of water.

Wrap the tube in a foil and incubate on ice for 45 min.

After the incubation, the reaction is then precipitated in:

| | | |
|---|---|---|
| 5M NaCl | 10 | μl |
| 20% SDS | 0.5 | μl |
| isopropanol | 61 | μl |

Incubate on ice for at least 30 min, then spin it down at max speed at 4° C. for 30 min and wash once with 70% ethanol and then once with 80% EtOH.

D. Biotinylation of the mRNA diol group

Resuspend the DNA in 110 μl DEPC treated water, then add the following reagents:

| | | |
|---|---|---|
| 20% SDS | 5 | μl |
| 2 M NaOAc pH 6.1 | 5 | μl |
| 10 mm biotin hydrazide (freshly made) | 300 | μl |

Wrap in a foil and incubate at room temperature overnight.

E. RNase I treatment

Precipitate DNA in:

| | | |
|---|---|---|
| 5M NaCl | 10 | μl |
| 2M NaOAc pH 6.1 | 75 | μl |
| biotinylated mRNA:cDNA | 420 | μl |
| 100% EtOH (2.5 Vol) | 1262.5 | μl |

(Perform this precipitation in two tubes and split the 420 μl of DNA into 210 μl each, add 5 μl of 5M NaCl, 37.5 μl of 2M NaOAc pH 6.1, and 631.25 μl of 100% EtOH).

Store at −20° C. for at least 30 min. Spin the DNA down at 4° C. at maximal speed for 30 min and wash twice with 80% EtOH, then dissolve DNA in 70 μl RNase free water. Pool two tubes and end up with 140 μl.

Add the following reagents:

| | | |
|---|---|---|
| RNase One 10U/μl | 40 | μl |
| 1^{st} cDNA:RNA | 140 | μl |
| 10X buffer | 20 | μl |

Incubate at 37° C. for 15 min.

Add 5 μl of 40 μg/μl yeast tRNA to each sample for capturing.

F. Full Length 1^{st} cDNA Capturing

Blocking the beads with yeast tRNA:

| | | |
|---|---|---|
| Beads | 1 | ml |
| Yeast tRNA 40 μg/μl | 5 | μl |

Incubate on ice for 30 min with mixing, wash 3 times with 1 ml of 2M NaCl, 50 mMEDTA, pH 8.0.

Resuspend the beads in 800 μl of 2M NaCl, 50 mMEDTA, pH 8.0, add RNase I treated sample 200 μl, and incubate the reaction for 30 min at room temperature.

Capture the beads using the magnetic stand, save the supernatant, and start the following washes:

2 washes with 2M NaCl, 50 m MEDTA, pH 8.0, 1 ml each time;
1 wash with 0.4% SDS, 50 μg/ml tRNA;
1 wash with 10 mm Tris-Cl pH 7.5, 0.2 m MEDTA, 10 mM NaCl,
20% glycerol;
1 wash with 50 μg/ml tRNA; and
1 wash with 1^{st} cDNA buffer.

G. Second Strand cDNA Synthesis

Resuspend the beads in:

| | | |
|---|---|---|
| *5X first buffer | 8 | μl |
| *0.1 mM DTT | 4 | μl |
| *10 mm dNTP mix | 8 | μl |
| *5X 2nd buffer | 60 | μl |
| *E. coli Ligase 10 U/μl | 2 | μl |
| *E. coli DNA polymerase 10 U/μl | 8 | μl |
| *E. coli RNaseH 2 U/μl | 2 | μl |
| P32 dCTP 10 μci/μl | 2 | μl |
| Water up to 300 μl | 208 | μl |

Incubate at 16° C. for 2 hr with mixing the reaction every 30 min.

Add 4 μl of T4 DNA polymerase and incubate for additional 5 min at 16° C.

Elute 2^{nd} cDNA from the beads.

Use a magnetic stand to separate the 2^{nd} cDNA from the beads, then resuspend the beads in 200 μl of water, and then separate again, pool the samples (about 500 μl).

Add 200 μl of water to the beads, then 200 μl of phenol:chloroform, vortex, and spin to separate the sample with phenol.

Pool the DNA together (about 700 μl) and use phenol to clean the DNA again. The DNA is then precipitated in 2 μg of glycogen and 0.5 vol of 7.5M NH₄OAc and 2 vol of 100% EtOH.

Precipitate overnight. Spin down the pellet and wash with 70% EtOH, air-dry the pellet.

| | | | | | |
|---|---|---|---|---|---|
| DNA | 250 | μl | DNA | 200 | μl |
| 7.5 M NH₄OAc | 125 | μl | 7.5 M NH₄OAc | 100 | μl |
| 100% EtOH | 750 | μl | 100% EtOH | 600 | μl |
| glycogen 1 μg/μl | 2 | μl | glycogen 1 μg/μl | 2 | μl |

H. Sal I Adapter Ligation

Resuspend the pellet in 26 μl of water and use 1 μl for TAE gel.

Set up reaction as follows:

| | | |
|---|---|---|
| 2^{nd} strand cDNA | 25 | μl |
| *5X T4 DNA ligase buffer | 10 | μl |
| *Sal I adapters | 10 | μl |
| *T4 DNA ligase | 5 | μl |

Mix gently, incubate the reaction at 16° C. overnight.

Add 2 μl of ligase on the second day and incubate at room temperature for 2 hrs (optional).

Add 50 μl water to the reaction and use 100 μl of phenol to clean the DNA, 90 μl of the upper phase is transferred into a new tube and precipitated in:

| | | |
|---|---|---|
| Glycogen 1 μg/μl | 2 | μl |
| Upper phase DNA | 90 | μl |
| 7.5 M NH₄OAc | 50 | μl |
| 100% EtOH | 300 | μl |

Precipitate at −20° C. overnight.

Spin down the pellet at 4° C. and wash in 70% EtOH, dry the pellet.

I. Not I Digestion

| | | |
|---|---|---|
| 2^{nd} cDNA | 41 | μl |
| *Reaction 3 buffer | 5 | μl |
| *Not I 15 u/μl | 4 | μl |

Mix gently and incubate the reaction at 37° C. for 2 hrs.

Add 50 μl of water and 100 μl of phenol, vortex , and take 90 μl of the upper phase to a new tube, then add 50 μl of NH₄OAc and 300 μl of EtOH. Precipitate overnight at −20° C.

Cloning, ligation, and transformation are performed per the Superscript cDNA synthesis kit.

### Example 3

This example describes cDNA sequencing and library subtraction.

Individual colonies can be picked and DNA prepared either by PCR with M13 forward primers and M13 reverse primers, or by plasmid isolation. cDNA clones can be sequenced using M13 reverse primers.

cDNA libraries are plated out on 22×22 cm² agar plates at a density of about 3,000 colonies per plate. The plates are incubated in a 37° C. incubator for 12-24 hours. Colonies are picked into 384-well plates by a robot colony picker, Q-bot (GENETIX Limited). These plates are incubated overnight at 37° C. Once sufficient colonies are picked, they are pinned onto 22×22 cm² nylon membranes using Q-bot. Each membrane holds 9,216 or 36,864 colonies. These membranes are placed onto an agar plate with an appropriate antibiotic. The plates are incubated at 37° C. overnight.

After colonies are recovered on the second day, these filters are placed on filter paper prewetted with denaturing solution for four minutes, then incubated on top of a boiling water bath for an additional four minutes. The filters are then placed on filter paper prewetted with neutralizing solution for four minutes. After excess solution is removed by placing the filters on dry filter papers for one minute, the colony side of the filters is placed into Proteinase K solution and incubated at 37° C. for 40-50 minutes. The filters are placed on dry filter papers to dry overnight. DNA is then cross-linked to the nylon membrane by UV light treatment.

Colony hybridization is conducted as described in Molecular Cloning: A Laboratory Manual, 2^{nd} Edition, supra. The following probes can be used in colony hybridization:

1. First strand cDNA from the same tissue as the library was made from to remove the most redundant clones;

2. 48-192 most redundant cDNA clones from the same library based on previous sequencing data;

3. 192 most redundant cDNA clones in the entire maize sequence database;

4. A Sal-A20 oligo nucleotide: TCG ACC CAC GCG TCC GAA AAA AAA AAA AAA AAA AAA, removes clones containing a poly A tail but no cDNA; and

5. cDNA clones derived from rRNA.

The image of the autoradiography is scanned into a computer and the signal intensity and cold colony addresses of each colony are analyzed. Re-arraying of cold-colonies from 384 well plates to 96 well plates is conducted using Q-bot.

### Example 4

This example describes identification of the gene from a computer homology search.

Gene identities can be determined by conducting BLAST (Basic Local Alignment Search Tool; Altschul, S. F., et al., (1993) supra; see also www.ncbi.nlm.nih.gov/BLAST/) searches under default parameters for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences are analyzed for similarity to all publicly available DNA sequences contained in the “nr” database using the BLASTN algorithm. The DNA sequences are translated in all reading frames and compared for similarity to all publicly available protein sequences contained in the “nr” database using the BLASTX algorithm (Gish, W. and States, D. J. Nature Genetics 3: 266-272 (1993)) provided by the NCBI. In some cases, the sequencing data from two or more clones containing overlapping segments of DNA are used to construct contiguous DNA sequences.

Sequence alignments and percent identity calculations can be performed using the Megalign program of the LASERGENE bioinformatics computing suite (DNASTAR Inc., Madison, Wis.). Multiple alignment of the sequences can be performed using the Clustal method of alignment (Higgins and Sharp (1989) CABIOS. 5: 151-153) with the default parameters (GAP PENALTY=10, GAP LENGTH PENALTY=10). Default parameters for pairwise alignments using the Clustal method are KTUPLE 1, GAP PENALTY=3, WINDOW=5 and DIAGONALS SAVED=5.

### Example 5

This example describes expression of transgenes in monocot cells.

A transgene comprising a cDNA encoding the instant polypeptides in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by PCR of the cDNA clone using appropriate oligonucleotide primers. Cloning sites (NcoI or SmaI) can be incorporated into the oligonucleotides to provide proper orientation of the DNA fragment when inserted into the digested vector pML103 as described below. Amplification is then performed in a standard PCR. The amplified DNA is then digested with restriction enzymes NcoI and SmaI and fractionated on an agarose gel. The appropriate band can be isolated from the gel and combined with a 4.9 kb NcoI-SmaI fragment of the plasmid pML103. Plasmid pML103 has been deposited under the terms of the Budapest Treaty at the ATCC (American Type Culture Collection, 10801 University Blvd., Manassas, Va. 20110-2209), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL1-Blue (Epicurian Coli XL-1 Blue; Stratagene). Bacterial transformants can be screened by restriction enzyme digestion of plasmid DNA and limited nucleotide sequence analysis using the dideoxy chain termination method (Sequenase DNA Sequencing Kit; U. S. Biochemical). The resulting plasmid construct would comprise a transgene encoding, in the 5′ to 3′ direction, the maize 27 kD zein promoter, a cDNA fragment encoding the instant polypeptides, and the 10 kD zein 3′ region.

The transgene described above can then be introduced into corn cells by the following procedure. Immature corn embryos can be dissected from developing caryopses derived from crosses of the Pioneer® inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al. Sci Sin Peking 18: 659-668 (1975)). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (Hoechst Ag, Frankfurt, Germany) or equivalent may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The Pat gene in p35S/Ac is under the control of the 35S promoter from Cauliflower Mosaic Virus (Odell et al. Nature 313: 810-812 (1985)) and the 3′ region of the nopaline synthase gene from the T-DNA of the Ti plasmid of Agrobacterium tumefaciens.

The particle bombardment method (Klein et al, supra) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton flying disc (Bio-Rad Labs). The particles are then accelerated into the corn tissue with a Biolistic PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covers a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains glufosinate (2 mg per liter) and lacks casein or proline. The tissue continues to grow slowly on this medium. After an additional 2 weeks the tissue can be transferred to fresh N6 medium containing glufosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2, 4-D. After two weeks the tissue can be transferred to regeneration medium (Fromm et al. (1990) Bio/Technology 8: 833-839).

### Example 6

This example describes expression of transgenes in dicot cells.

A seed-specific expression cassette composed of the promoter and transcription terminator from the gene encoding the β subunit of the seed storage protein phaseolin from the bean Phaseolus vulgaris (Doyle et al (1986) J Biol Chem 261: 9228-9238) can be used for expression of the instant polypeptides in transformed soybean. The phaseolin cassette includes about 500 nucleotides upstream (5′) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), SmaI, KpnI and XbaI. The entire cassette is flanked by Hind III sites.

The cDNA fragment of this gene may be generated by PCR of the cDNA clone using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC18 vector carrying the seed expression cassette.

Soybean embryos may then be transformed with the expression vector comprising sequences encoding the instant polypeptides. To induce somatic embryos, cotyledons, 3-5 mm in length are dissected from surface sterilized, immature seeds of the soybean cultivar A2872, and can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures are maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Klein et al., supra; U.S. Pat. No. 4,945,050). A DuPont Biolistic PDS1000/HE instrument (helium retrofit), available from Bio-Rad Laboratories, Hercules, Calif., can be used for these transformations.

A selectable marker gene which can be used to facilitate soybean transformation is a transgene composed of the 35S promoter from Cauliflower Mosaic Virus (Odell et al., supra), the hygromycin phosphotransferase gene from plasmid pJR225 (from E. coli; Gritz et al. Gene 25: 179-188 (1983)) and the 3′ region of the nopaline synthase gene from the T-DNA of the Ti plasmid of Agrobacterium tumefaciens. The seed expression cassette comprising the phaseolin 5′ region, the fragment encoding the instant polypeptide and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μl spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 7

This example describes expression of a transgene in microbial cells.

The cDNAs encoding the instant polypeptides can be inserted into the T7 E. coli expression vector pBT430. This vector is a derivative of pET-3a (Rosenberg et al., Gene 56: 125-135 (1987)) which employs the bacteriophage T7 RNA polymerase/T7 promoter system. Plasmid pBT430 was constructed by first destroying the EcoRI and HindIII sites in pET-3a at their original positions. An oligonucleotide adaptor containing EcoRI and HindIII sites was inserted at the BamHI site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3aM in this region, 5′-CATATGG, was converted to 5′-CCCATGG in pBT430.

Plasmid DNA containing a cDNA may be appropriately digested to release a nucleic acid fragment encoding the protein. This fragment may then be purified on a 1% NuSieve GTG low melting agarose gel (FMC). The buffer and agarose contain 10 μg/ml ethidium bromide for visualization of the DNA fragment. The fragment can then be purified from the agarose gel by digestion with GELase (Epicentre Technologies) according to the manufacturer's instructions, ethanol precipitated, dried and resuspended in 20 μL of water. Appropriate oligonucleotide adapters may be ligated to the fragment using T4 DNA ligase (New England Biolabs, Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as described above. The prepared vector pBT430 and fragment can then be ligated at 16° C. for 15 hours followed by transformation into DH5 electrocompetent cells (GIBCO BRL). Transformants can be selected on agar plates containing LB media and 100 μg/mL ampicillin. Transformants containing the gene encoding the instant polypeptides are then screened for the correct orientation with respect to the T7 promoter by restriction enzyme analysis.

For high level expression, a plasmid clone with the cDNA insert in the correct orientation relative to the T7 promoter can be transformed into E. coli strain BL21 (DE3) (Studier et al. J Mol Biol 189:113-130 (1986)). Cultures are grown in LB medium containing ampicillin (100 mg/L) at 25° C. At an optical density at 600 nm of approximately 1, IPTG (isopropylthio-β-galactoside, the inducer) is added to a final concentration of 0.4 mM and incubation is continued for 3 h at 25°. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One microgram of protein from the soluble fraction of the culture can be separated by SDS-polyacrylamide gel electrophoresis. Gels can be observed for protein bands migrating at the expected molecular weight.

### Example 8

This example describes the use of CuraGen mRNA profiling technology to aid in the discovery of the genes of the present invention.

Companies such as CuraGen Corp. (New Haven Conn.) provide robust expression profiling based upon modified differential display techniques. See, e.g., WO 97/15690, which is herein incorporated by reference. Accordingly, one of skill can have expression profiling performed by companies which specialize in such techniques.

The mRNA profiling was done using the CuraGen GeneCalling™ technology (U.S. Pat. No. 5,871,697; Shimkets et al. Nature Biotechnology 17: 798-803 (1999); Bruce et al. Plant Cell 12: 65-80 (2000)). In brief, this technology employs a genome-wide high-throughput mRNA differential display of PCR-amplified restriction enzyme digested cDNA fragments separated by size through slab gel or capillary electrophoresis. A total of 48 distinct restriction enzyme pair combinations were used for this study. Gene identities can be made by comparing the patterns of coordinately-expressed cDNA fragments to computer-generated virtual restriction enzyme digests of cDNA sequence datasets. At the time of this profiling (early 1999) the sequence dataset involved circa 350,000 Ests proprietary to DuPont/Pioneer supplemented with available public sequences. Gene identities can be further affirmed by direct cloning and sequencing, or by a competitive-PCR reaction that involves a reamplification of the sample in the presence of unlabeled oligonucleotide primers designed from the candidate gene sequence, but 10 nts internal from the original restriction sites defining the cDNA fragment. If the gene is correctly identified, then the cDNA fragment in the competitive-PCR reaction is not labeled and thus appears absent.

Following below are descriptions of the key disease related experiments wherein defense-related differential expression was observed. For each of these experiments total RNA was isolated from about 4 g of tissue by the Tri-Reagent method (Molecular Research Center, Cincinnati, Ohio, U.S.A.).

Bipolaris maydis inoculation. Previously known as Helminthosporium maydis, B. maydis is the anamorph form of Cochiobolus heterostrophus, an ascomycete pathogen that is the causal agent of Southern corn leaf blight (White, Compendium of Corn Diseases, American Phytopathological Press, St. Paul, Minn. (1999)). Maize plants, either wildtype genotype (mostly A632 background) susceptible to B. maydis, or rhmi genotype, resistant to B. maydis, were grown in pots in the greenhouse. The growth conditions, B. maydis source, and inoculation conditions were essentially as described in Simmons et al., 1998 (Mol Plant Microbe In 11: 1110-1118). Samples were collected 24 hrs after inoculation, mRNA production, and subsequent analysis was as described in Simmons et al., 2001 (Mol Plant Microbe In 14: 947-954).

In particular, Cochliobolus heterostrophus (Drechs.) Drechs. Race 0 (anamorph: Bipolaris maydis; causal agent of southern corn leaf blight) isolate TX001, was obtained from field sources at Pioneer Hi-Bred and maintained on potato dextrose agar medium or for long-term storage in silica gel as described in Dhingra and Sinclair 1995, Basic Plant Pathology Methods, 2^{nd} ed. Lewis Publishers, Boca Raton, Fla. Puccinia sorghi Schwein. (causal agent of common rust) isolate PS001, was obtained from field sources at Pioneer Hi-Bred and maintained on B73 inbred seedling leaves essentially as described in Dhingra and Sinclair 1995, supra. For general leaf inoculation, spore suspensions of 4×10⁴ per ml of 0.02% Tween 20 were sprayed as an aerosol on the leaves. Approximately 0.5 ml was applied per leaf late in the afternoon. The plants were then immediately covered with a plastic tent and kept at room temperature in order to enhance humidity and spore germination. The plastic tent was removed early in the morning, and the plants were returned to the greenhouse for the duration of the experiment. For whorl inoculation with C. heterostrophus, 0.2 ml of 4×10⁴ spores per ml of 0.02% Tween 20 was deposited in the whorl. After the rust inoculation, the plants were moved to a growth chamber (14-h day, 27° C., 80 to 90% relative humidity, 200 to 300 μE s⁻1 m⁻² from both fluorescent and incandescent lamps) to avoid rust contamination of the greenhouse. Leaf tissues were collected and frozen 24 h postinoculation. Individual plants were scored for the rhm1 phenotype 96 h postinoculation, after which the frozen rhm1 or wild-type tissues, control or inoculated, from at least six rhm1 plants and up to 18 wild-type plants, were then pooled separately. Total RNA from each pool was isolated from 4 g of leaf powder by the Tri-Reagent method (Molecular Research Center, Cincinnati, Ohio, U.S.A.) and sent to CuraGen for analysis. The results were evaluated and analyzed with CuraGen GeneScape software.

Transgenic avrRxv expression. The avirulence gene avrRxv from Xanthomonas campestris pv. vesicatoria causes incompatible resistant reactions in numerous dicots and monocot plants, including maize (Whalen et al, Proc Natl Acad Sci USA 85: 6743-6747 (1988)), and is part of a family of proteins with possible protease function that cause disease reactions in both plants and animals including humans (see Orth et al. Science 290: 1594-1597 (2000)). Control and experimental maize Hi-II embryo-derived cell suspensions, transgenic for an estradiol responsive ERE promoter construct driving expression of the avirulence gene avrRxv, were produced as described (Briggs et al., WO 99/43823 (1999); Simmons et al., Maize Genetics Cooperation Newsletter 76 (2002)). RNA was harvested for analysis either 4 or 24 hrs after estradiol treatment.

Les9 disease lesion mimic. The disease lesion mimic Les9 is a partially dominant genetic background that forms spontaneous lesions similar to a disease response (Hoisington, Maize Genetics Cooperation Newsletter 60: 50-51 (1986)), and such plants exhibit enhanced resistance to B. maydis and elevated PR protein expression (Yalpani and Fridlender, unpublished data). Les9 and wildtype control plant tissue was grown and harvested as described in Nadimpalli et al., 2000 (J Biol Chem 38: 29579-29586) from a ‘family 2’ which was not yet exhibiting Les9 lesions (pre-initiation stage), and from a ‘family 6’ (pedigree Mo95-09 Les9, from Les9×br2hm1hm2), that was experiencing Les9 lesion formation (post-initiation stage).

Ultraviolet light. Ultraviolet light is known to induce various pathogenesis-related proteins in plants (eg. Brederode, Plant Mol Biol 17: 1117-1126 (1991)), including maize (Didierjean, Planta 199: 1-8 (1996)). For this experiment greenhouse-grown B73 genotype V2-V3 seedlings were horizontally irradiated for 30 min with a total dose of 782 mJ/cm2 of UV-C light (germicidal lights). Seedlings were rotated 90 degrees four times during irradiation to get even exposure. Twelve hours later irradiated and control leaf tissue, minus midrib, was collected, frozen in liquid nitrogen, and stored at −80° C. prior to RNA extraction. No visible symptoms of irradiation were apparent.

Cochliobolus carbonum inoculation. The C. carbonum ascomycete is the causal agent of maize leaf spot, and its pathogenicity is determined by a cyclic tetrapeptide HC-toxin (Scheffer et al., Phytopathology 57: 1288-1291 (1967)). Strains lacking HC-toxin production (tox minus) are not generally virulent. Maize resistance to C. carbonum is determined by the Hm1 gene that encodes a reductase that degrades the HC-toxin (Johal and Briggs, Science 258: 985-987 (1992)), and to a lesser extent by the related Hm2 gene. Maize strains, such as Pr, that lack functional Hm1 and Hm2 genes are susceptible to C. carbonum (Meeley et al., Plant Cell 4: 71-77 (1992)). Maize Pr genotype greenhouse-grown V2-V3 seedlings were inoculated with either C. carbonum tox minus (Briggs isolate 26.R.4), HC-toxin alone, or C. carbonum tox minus plus HC-toxin. The C. carbonum inoculation involved spray inoculation of 4×10⁴ conidiaspores/ml, and was performed essentially as the B. maydis inoculation described in Simmons et al., 1998. The HC-toxin was prepared at Pioneer, and applications were at 5 μg/ml in the spray inoculant. Tissue samples were harvested either 6 or 22 hrs after inoculation.

Transgenic induced flavonoid biosynthesis. Flavonoids are a complex group of metabolites found in plants that have various functions, among them defense against pathogens (Koes et al., BioEssays 16: 123-132 (1994)). Flavonoid production is frequently induced in plant defense reactions, and some have been implicated as determinants of disease resistance, including maize (eg. Lee et al., Biochem 28: 2540-2544 (1989)). Maize BMS cells were engineered to have chemically-inducible expression of the trans-activator genes for maize flavonoid biosynthesis C1+R or P. The experimental design and tissue preparation was as described in Bruce et al., supra.

Using CuraGen mRNA profiling technology an mRNA band was identified in a study involving Cochliobolus heterostrophus inoculation of leaves that was markedly upregulated in inoculated leaves versus control. Subsequent analysis of all these inoculations involving C. heterostrophus revealed that it was upregulated in all such inoculations. This indicated a consistency of response.

The experiment involving avrRxv induction (the ERE-avrRxv defense activation studies; WO 99/43823) also revealed that this band was upregulated. It was one of a few bands co-induced between the two studies, and indicated that this band (and the gene it represents) is a good indicator of a defense response. Further analysis revealed that this band was upregulated in diverse defense-related experiments as described above, including, the les9 disease lesion mimic studies, the Cochliobolus carbonum inoculation of leaves studies, and the ultraviolet light treatment. It was also upregulated in experiments involving artificially induced activation of the flavonoid biosynthetic system. It was upregulated in few other experiments, indicating that it was a gene whose expression is strongly and exclusively associated with a defense response in maize. No other band is known to show such a consistent pattern at this time. Only a few genes, such as a few chitinases, show strong and consistent defense activation. The band was requested for isolation from the les9 study. The band was successfully isolated and the sequence showed a match to several proprietary ESTs, the longest of which was p0018.chsth71r (SEQ ID NO: 1). This clone was ordered, sequenced to completion, and analyzed.

Table 3 shows the results of the differential mRNA expression studies for SEQ ID NO: 1 as described above in the various defence-related experiments.

TABLE 3

| Experiment Description | Fold^{a} | SE^{b} | N^{c} |
|---|---|---|---|
| Bipolaris maydis | | | |
| Experiment 1, wt, infected vs uninfected, 24 hrs | 11.4 | 1.0 | 4 |
| Experiment 2, wt, infected vs uninfected, 24 hrs | 7.6 | 2.8 | 5 |
| Experiment 3, wt, infected vs uninfected, 24 hrs | 13.2 | 6.6 | 5 |
| Experiment 1, infected, wt vs rhm1, 24 hrs | 1.1 | 0.2 | 5 |
| Experiment 2, infected, wt vs rhm1, 24 hrs | 1.0 | 0.1 | 5 |
| Experiment 3, infected, wt vs rhm1, 24 hrs | 1.0 | 0.1 | 5 |
| Cochliobolus carbonum | | | |
| Toxin minus strain plus HC-toxin vs uninfected, 6 hrs | 1.8 | 0.2 | 4 |
| Toxin minus strain plus HC-toxin vs uninfected, 22 hrs | 4.3 | 0.8 | 4 |
| Toxin minus strain vs uninfected, 6 hrs | 1.2 | 0.2 | 4 |
| Toxin minus strain vs uninfected, 22 hrs | 5.4 | 1.9 | 3 |
| HC-toxin only vs untreated, 6 hrs | 1.0 | 0.1 | 4 |
| HC-toxin only vs treated, 22 hrs | 2.5 | 0.9 | 3 |
| Les9 disease lesion mimic | | | |
| Les9 vs wt, pre-initiation | 6.2 | 1.5 | 5 |
| Les9 vs wt, post-initiation | 8.5 | 2.9 | 4 |
| Ultraviolet light | | | |
| Treated vs untreated, 12 hrs | 6.1 | 0.8 | 4 |
| Chemically-induced avrRxv expression | | | |
| Induced vs uninduced, 4 hrs | 1.4 | 0.4 | 4 |
| Induced vs uninduced, 24 hrs | 3.1 | 0.6 | 4 |
| Chemically-induced flavonoid synthesis | | | |
| CRC genes construct, 6 vs 0 hrs | 1.8 | 0.3 | 3 |
| CRC genes construct, 24 vs 0 hrs | 2.5 | 0.5 | 3 |
| P gene construct, 6 vs 0 hrs | 1.0 | 0.1 | 3 |
| P gene construct, 24 vs 0 hrs | 0.9 | 0.0 | 3 |
| Control, 6 vs 0 hrs | 1.0 | 0.3 | 3 |
| Control, 24 vs 0 hrs | 0.8 | 0.1 | 3 |

^{a}Average fold change of between 3-5 cDNA fragments from Zm-mfs1 that were assayed. All calculations involved the following five two-restriction enzyme digested cDNA fragments: m110-301 (MfeI-BspEI) and d010-205 (AcsI-BspEI), with i0r0-179 (BglII-EcoRI), w0i0-129 (NheI- BglII), w0i0-358 (NheI- BglII).
^{b}Standard error of the fold changes for the cDNA fragments used in the calculation.
^{c}Number of the five cDNA fragments for Zm-mfs1 that were used in the calculation. Some were not used because useful expression results were not available.

### Example 9

This example describes the determination of the nucleic acid sequences coding for defense inducible genes (DIGs) of the present invention and in particular for SEQ ID NOs: 1 and 2.

Specifically, SEQ ID NO: 1 was compared to the GenSeq database (Derwent; Alexandria, Va.) using BLASTP 2.0.4 (Altschul, et al. (1990) supra). GSP:R47339 (Accession number AAR47388), which codes for a peptide fragment of a multi-drug resistance transporter protein, displayed a 26% sequence identity to SEQ ID NO: 1. Additionally, three Arabidopsis peptide fragments (Accession Nos. AAG23007, AAG23008, and AAG23009) respectively have 51.3%, 49.7%, and 48.4% sequence identity to SEQ ID NO: 2. These three Arabidopsis proteins, although differing at the N-terminus, each encode an identical protein. While this Arabidopsis protein is referred to in the database as a signal transduction protein, careful analysis showed that it has conserved regions in all the key sites (see Table 5) indicative of a multifacilitator super family protein of the subfamily containing multidrug efflux transporters.

A BLASTN search identified as the closest match to SEQ ID NO: 1 an Oryza sativa EST (SEQ ID NO: 7, GB accession no. C26087). PSORT (protein sorting and protein translocation prediction analyses) and SIGNALP (signal peptide prediction analysis) of SEQ ID NO: 2 suggested that the protein encoded by this sequence was transmembranous. Transit peptide prediction indicates a transit peptide of appropriate length and a good cleavage site.

Table 4 shows the relationship of SEQ ID NO: 2 to its closest homologs ordered by decreasing amino acid identity. Table 5 shows the key conserved domains, containing MFS and antiporter motifs, of SEQ ID NO: 2 compared to those of its closest homologs listed in Table 4. It also shows the distinction of the plant subfamily in the transmembrane (TM) TM-8-TM-9 loop and TM-7 domains.

TABLE 4

| Species (Gene) | Accession | AA | ID | Sim |
|---|---|---|---|---|
| Z.mays (SEQ ID NO: 2) | gi\|15796516 | 488 | 100 | 100 |
| O. sativa (SEQ ID NO: 7) | Pending | 497 | 84.1 | 88.5 |
| O. sativa | gi\|6498423 | 556 | 64.3 | 69.3 |
| O. sativa | gi\|6630695 | 398 | 59.9 | 68.7 |
| O. sativa (SEQ ID NO: 8) | Pending | 470 | 54.7 | 63.0 |
| Z. mays (SEQ ID NO: 6) | Pending | 399 | 54.5 | 61.5 |
| A. thaliana | gi\|11358901 | 479 | 52.1 | 62.7 |
| A. thaliana | gi\|10177340 | 441 | 51.4 | 61.6 |
| A. thaliana | gi\|10177339 | 515 | 45.5 | 56.6 |
| E. coli (Tn10, TetA) | gi_43701 | 401 | 26.1 | 39.8 |
| S. aureus (NorA) | gi_4115707 | 388 | 25.8 | 37.8 |
| B. subtilis (blt) | gi_2635104 | 400 | 25.3 | 35.2 |
| S. pneumoniae | gi_3820455 | 399 | 25.3 | 32.6 |
| E. coli | gi_4062627 | 408 | 25.1 | 34.7 |
| S. cerevisiae | gi\|10383787 | 611 | 24.7 | 36.1 |
| B. subtilis (BMR) | gi_142606 | 389 | 24.4 | 37.3 |
| P. mirabilis | gi_4104705 | 398 | 23.4 | 35.3 |
| A. tumefaciens (TetA) | gi_3860032 | 394 | 22.6 | 33.4 |

TABLE 5

| Species (Gene) | TM2-TM3 Loop | TM-5, Antiporter | TM8-TM9 Loop | TM-7, HD |
|---|---|---|---|---|
| Z. mays (Zm-Mfs1) | GMFADKYGRK | SLVTSSRAIALVIGPALVIGAIGG | AKYFGPIKTFRP | FSMHDTAY |
| (SEQ ID NO: 2) | (SEQ ID NO: 13) | (SEQ ID NO: 14) | (SEQ ID NO: 15) | (SEQ ID NO: 16) |
| O. sativa (Os-Mfs1) | GIFADKYGRK | SLVTSSRAIALVVGAIGG | AKYVGPIKPFRY | FSLHDTAY |
| (SEQ ID NO: 7) | (SEQ ID NO: 17) | (SEQ ID NO: 18) | (SEQ ID NO: 19) | (SEQ ID NO: 20) |
| O. sativa | n/a | SLVTSSRAIALVVGPAIGG | KYVGPIKPFRY | FSLHDTAY |
| | | (SEQ ID NO: 21) | (SEQ ID NO: 22) | (SEQ ID NO: 23) |
| O. sativa | GIVADKYGRK | SLVSSSRGIGLIVGPAIGG | AKSVEPITLVRI | FSLQDVAY |
| | (SEQ ID NO: 24) | (SEQ ID NO: 25) | (SEQ ID NO: 26) | (SEQ ID NO: 27) |
| O. sativa (Os-Mfs2) | GMVADRIGRK | SIVSTAWGIGLVVGPATGG | DKILGPIHSTRI | FSLHDTAY |
| (SEQ ID NO: 8) | (SEQ ID NO: 28) | (SEQ ID NO: 29) | (SEQ ID NO: 30) | (SEQ ID NO: 31) |
| Z. mays (Zm-Mfs2) | GVVADRVGRK | SVVSTAWGMGVIIGPAIGG | NKILGPVNSTRV | FSLHDTAY |
| (SEQ ID NO: 6) | (SEQ ID NO: 32) | (SEQ ID NO: 33) | (SEQ ID NO: 34) | (SEQ ID NO: 35) |
| A. thaliana | GKLADRYGRK | SVVSTSRGIGLILGPAIGG | EKSVGLLAVIRL | FSLQEIAY |
| | (SEQ ID NO: 36) | (SEQ ID NO: 37) | (SEQ ID NO: 38) | (SEQ ID NO: 39) |
| A. thaliana | GLVADRYGRK | SAVSTAWGIGLIIGPAIGG | ERLLGPIIVTRI | FSLHDMAY |
| | (SEQ ID NO: 40) | (SEQ ID NO: 41) | (SEQ ID NO: 42) | (SEQ ID NO: 43) |
| A. thaliana | GIVADRYGRK | SAVSTAWGIGLIIGPALGG | EKLLGPVLTRY | LCLHDTAY |
| | (SEQ ID NO: 44) | (SEQ ID NO: 45) | (SEQ ID NO: 46) | (SEQ ID NO: 47) |
| E. coli (Tn10, TetA) | GKMSDRFGRR | GWLGASFGLGLIAGPIIGG | GRIATKWGEK | AQLIGQIP |
| | (SEQ ID NO: 48) | (SEQ ID NO: 49) | (SEQ ID NO: 50) | (SEQ ID NO: 51) |
| S. aureus (NOrA) | GTLADKLGKK | GYMSAIINGFILGPCIGG | DKFMYFSEL | LAFGLSAF |
| | (SEQ ID NO: 52) | (SEQ ID NO: 53) | (SEQ ID NO: 54) | (SEQ ID NO: 55) |
| B. subtilis (blt) | GRWVDRFGRK | GYVSAAISTGFIIGPCAGG | GKLVNKLGEK | MAFGLSAY |
| | (SEQ ID NO: 56) | (SEQ ID NO: 57) | (SEQ ID NO: 58) | (SEQ ID NO: 59) |
| S. pneumoniae | GILADKYGRK | GKLGDKVGNH | GKLGDKVGNH | IQFSAQSI |
| | (SEQ ID NO: 60) | (SEQ ID NO: 61) | (SEQ ID NO: 62) | (SEQ ID NO: 63) |
| E. coli | GGLADRKGRK | GTLSTGGVSGALLGPMAGG | GKLGDRIGEP | IQVATGSI |
| | (SEQ ID NO: 64) | (SEQ ID NO: 65) | (SEQ ID NO: 66) | (SEQ ID NO: 67) |
| S. cerevisiae | GRFSEKHGRK | STMPLLFQFGAVVGPMIGG | DRNFDCLTIFRT | MALHLIVY |
| | (SEQ ID NO: 68) | (SEQ ID NO: 69) | (SEQ ID NO: 70) | (SEQ ID NO: 71) |
| B. subtilis (BMR) | GRWVDRFGRK | GYMSAAISTGFIIGPCIGG | DRFTRWFGEI | SSFGLASF |
| | (SEQ ID NO: 72) | (SEQ ID NO: 73) | (SEQ ID NO: 74) | (SEQ ID NO: 75) |
| P. mirabilis | GKLSDKYGRK | GFLGGAFGVGLIIGPMLGG | GKLAQKWGER | IQLIGQIP |
| | (SEQ ID NO: 76) | (SEQ ID NO: 77) | (SEQ ID NO: 78) | (SEQ ID NO: 79) |
| A. tumefaciens (TetA) | GALSDRFGRR | GTVGAVMSLGFIIGPVIGG | GPLSRRFGDL | FGLVAAIP |
| | (SEQ ID NO: 80) | (SEQ ID NO: 82) | (SEQ ID NO: 83) | (SEQ ID NO: 83) |

Transmembrane analysis indicates that SEQ ID NO: 2 is a transmembranous protein. Its N-terminus is predicted to be cytosolic, as is its C-terminus, and thus both ends are on the same side of the membrane. It crosses the membrane 12 times. The result is a protein with two cytosolic ends plus five external loops. In addition it has six external loops. This is the same topology for precedent MFS proteins, most of which are 12-TM proteins with some being 14-TM (reviewed in Van Bambeke et al., Biochemical Pharmacology 60: 457-470 (2000)). Furthermore, the Zm-Mfs1 protein possesses the characteristic MFS family signature sequence GX₃D(R/K)XGR(R/K) (see Maiden et al., Nature 325: 641-643 (1987); Yamaguchi et al., J Biol Chem 268: 6496-6504(1992)), located between the second and third transmembrane domains, which is thought to be involved in a general transport function of this protein superfamily, although not necessarily in substrate specificity (Yamaguchi et al., Id.).

MFS proteins with antiporter function possess a conserved motif GX₈GX₃GPX₂GG located in the fifth transmembrane domain (Varela et al. Mol Membr Biol 12: 313-319, 1995). The SEQ ID NO: 2 protein has the very closely-related sequence SX₈AX₃GPX₂GG at the same location, indicating that it is most closely related to the MFS antiporter efflux proteins of MFS families 1 and 2, which includes drug efflux proteins (Varela et al., Id.). Aside from the set of closely-related unknown plant genes, the global protein similarity of Zm-Mfs1 was highest to E. coli TetA(B) and S. aureus NorA (Tables 4 and 5), both of which are classified in MFS antiporters family 1.

The family of plant genes related to SEQ ID NO: 2 all have the positively-charged motif GX₃D(R/K)XGR(R/K) in the TM2-TM3 cytoplasmic loop (Table 5), which is characteristic of MFS genes (Maiden et al., supra.). The plant genes also have a motif in the fifth TM related to the MFS antiporter family motif GX₈GX₃GPX₂GG (Varela et al, supra.), however the plant genes follow the slightly modified expression SX₈(GA)X₃GPX₂GG (Table 5). It has been noted that substitutions of alanine and serine at the first two conserved glycine locations of this motif are acceptable variants that retain MFS antiporter protein activity (Varela et al., 1995). The TM8-TM9 cytoplasmic loop is not highly conserved between the bacterial genes, which follow the general expression (GD)(KR)X₅GX₂, and the plant genes, which follow the general expression X(KR)X₂GP(IV)X₃RX. The plant TM8-TM9 cytoplasmic loop has a net positive charge, especially for SEQ ID NO: 2 and its most-closely related proteins (net charge+3). Together, these domain differences indicate that the plant genes comprise a new subfamily of MFS genes. Among the non-plant genes, the yeast gene is most similar in the TM5 antiporter motif, and in the TM8-TM9 cytoplasmic loop (Table 5).

Both the E. coli TetA and LacK MFS genes have been shown to have single His residues located in TM8 and TM10 respectively, and each such His appears to be important for proton translocation and transport function (Yamaguchi et al., Biochem 35: 4359-4364 (1996); Püittner et al., Biochem 28: 2525-2533, (1989)). Moreover, acidic residues Glu or Asp that are proximal to these TM-located histidines have been implicated in the proton translocation coupled transport and substrate binding (Kimura and Yamaguchi, FEBS Letters 388: 50-52, (1996); Carrasco et al. Biochem 28: 2533-2539, (1989); Lee et al. (1989), supra. The plant genes do not have His conserved at these TM locations. Interestingly, while Zm-Mfs1 and others of the plant proteins do not have a single His conserved in TM8 or TM 10, but instead they have a single TM-located His in TM7. Importantly, this His is adjacent to a conserved acidic residue, usually Asp (Table 5). This Asp is the only acidic residue located in the middle of any of the 12 Zm-Mfs1 TM domains. The sequence homology surrounding this ‘HD’ motif is conserved in the plant TM7s, but the TM7 region is variable in the bacterial genes, suggesting a functional constraint on TM7 in the plant genes. Two of the plant genes have a Glu substitution for His, which despite their chemical differences, are both polar amino acids.

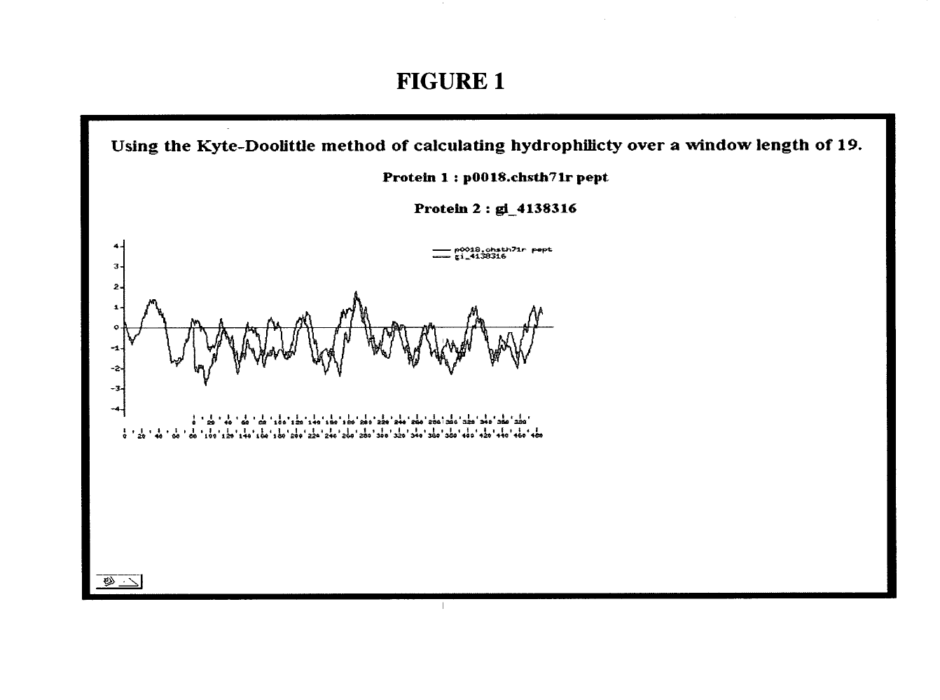

Kyte-Doolittle Hydrophobicity comparison between the maize gene p0018.chsth71r peptide (SEQ ID NO: 2) and that of a multidrug resistance protein from Pasteurella haemolytica demonstrates a striking similarity of hydrophobicity profiles to this example of a multidrug resistance efflux protein (FIG. 1). This analysis extends the sequence similarity comparison to indicate that this novel maize gene is related to multidrug resistance efflux proteins.

This analysis indicates that this maize gene is novel. However, it does have some limited sequence similarity to various transmembranous proteins, including those from bacteria and eukaryotes, such as fungi, which are multidrug resistance efflux proteins. As such, this might be its general function. However, it appears not to have been previously reported for maize. There are three closely related EST sequences, one in corn and two in nice. There are more distantly related maize ESTs in the public domain. The first rice clone rds1f.pk002.a8 (SEQ ID NO: 7) is of particular interest and has been completely sequenced, showing an 84% amino acid identity with SEQ ID NO: 1, and 88.5% similarity. SEQ ID NO: 7 has a methionine start codon in approximately the same position as does the maize gene represented by p0018.chsth71r (SEQ ID NO: 1, 2). However, of significance is three inframe stop codons immediately upstream from the methionine start codon. This indicates that the rice gene is full-length and that the maize gene of the present invention is also full-length.

In bacteria and some other organisms multidrug resistance efflux transporters are involved in exporting antibiotics. In this way the bacteria are rendered resistant to the antibiotics. In animals such multidrug resistance efflux transporter genes in cancerous cells result in resistance of those cancer cells to chemotherapeutic drugs. These genes may have other functions in effluxing cellular compounds that may be adaptive, such as toxins to pathogens of that organism.

Our observation that a novel gene in maize (SEQ ID NOs: 1 and 2) related to these multidrug resistance efflux transporters is induced in expression in response to diverse conditions associated with a defense response, suggests at least two explanations for the gene's adaptive function. The first is that this gene is part of a general defense response that helps guard the plant against antibiotics and compatibility factors produced by a pathogen. In this way the plant can shield itself from harm and colonization by the pathogen. According to this scenario, these genes may find utility in reducing the levels of pathogen-derived toxins, such as fungal toxins, that are often produced by fungal pathogens. In maize, such toxins are often associated with ear molds. In endeavoring to improve disease resistance, this invention may have the added benefit of reducing pathogen-derived toxins in food and feed derived from crop plants such as maize. In the second scenario the function of this and closely related genes is to efflux from plant cells metabolites that are antibiotic to pathogens. As such this is a strategy by the plant to thwart pathogen attack by creating an antibiotic barrage against the pathogens. Both of these scenarios may function in combination or simultaneously.

The above examples are provided to illustrate the invention but not to limit its scope. Other variants of the invention will be readily apparent to one of ordinary skill in the art and are encompassed by the appended claims. All publications, patents, patent applications, and computer programs cited herein are hereby incorporated by reference.

What is claimed is:

1. An isolated nucleic acid comprising a nucleotide sequence selected from the group consisting of:
(a) a nucleotide sequence set forth in SEQ ID NO: 1, 3, 4, 5, 6, 7, 8, and 9;
(b) a nucleotide sequence that encodes a polypeptide having an amino acid sequence set forth in SEQ ID NO:2, 10, 11, and 12;
(c) a nucleotide sequence amplified from a maize, rice, or wheat nucleic acid library which hybridizes, under stringent hybridization conditions, to a nucleotide sequence having a sequence set forth in SEQ ID NOs: 1, 3, 4, 5, 6, 7, 8, and 9;
(d) a nucleotide sequence which hybridizes under stringent conditions to a nucleotide sequence having a sequence set forth in SEQ ID NOs: 1, 3, 4, 5, 6, 7, 8, and 9;
(e) a nucleotide sequence characterized by at least 80% sequence identity to a nucleotide sequence set forth in SEQ ID NOs: 1, 3, 4, 5, 6, 7, 8, and 9;
(f) a nucleotide sequence characterized by at least 85% sequence identity to a nucleotide sequence set forth in SEQ ID NOs: 1, 3, 4, 5, 6, 7, 8, and 9;
(g) a nucleotide sequence characterized by at least 90% sequence identity to the nucleotide sequence set forth in SEQ ID NOs: 1, 3, 4, 5, 6, 7, 8, and 9;
(h) a nucleotide sequence that comprises the complement of any one of (a), (b), (c), or (d); and
(i) a nucleotide sequence comprising at least 100 contiguous nucleotides from a nucleotide sequence of (a), (b), (c), (d), (e), (f), or (g).

2. A DNA construct comprising a nucleotide sequence of claim 1, wherein said nucleotide sequence is operably linked, in sense or anti-sense orientation, to a promoter that drives expression in a host cell.

3. An expression cassette comprising the DNA construct of claim 2.

4. A host cell, having stably incorporated into its genome at least one DNA construct of claim 2.

5. The host cell of claim 4, wherein said host cell is a plant cell.

6. A plant having stably incorporated into its genome the DNA construct of claim 2.

7. The plant according to claim 6, wherein said plant is a monocot.

8. The plant according to claim 6, wherein said plant is a dicot.

9. The plant of claim 6, wherein said plant is selected from the group consisting of: maize, soybean, sunflower, sorghum, canola, wheat, alfalfa, cotton, rice, barley, and millet.

10. A transformed seed from the plant of claim 6.

11. An isolated polypeptide selected from the group consisting of:
(a) a polypeptide comprising an amino acid sequence set forth in SEQ ID NO: 2, 10, 11, and 12;
(b) a polypeptide characterized by at least 80% sequence identity to an amino acid sequence set forth in SEQ ID NO: 2, 10, 11, and 12;
(c) a polypeptide characterized by at least 85% sequence identity to an amino acid sequence set forth in SEQ ID NO: 2, 10, 11, and 12;
(d) a polypeptide characterized by at least 90% sequence identity to an amino acid sequence set forth in SEQ ID NO: 2, 10, 11, and 12; and
(e) a polypeptide characterized by at least 95% sequence identity to an amino acid sequence set forth in SEQ ID NO: 2, 10, 11, and 12.

12. A method of modulating the level of a defense induced gene expression in a plant cell, wherein the method comprises:
(a) introducing into a plant cell a DNA construct comprising a nucleotide sequence operably linked, in a sense or anti-sense orientation, to a promoter that drives expression in a host cell and said nucleotide sequence is selected from the group consisting of:
(1) a nucleotide sequence set forth in SEQ ID NO: 1, 3, 4, 5, 6, 7, 8, and 9;
(2) a nucleotide sequence that encodes a polypeptide having an amino acid sequence set forth in SEQ ID NO: 2, 10, 11, and 12;
(3) a nucleotide sequence amplified from a maize, rice, or wheat nucleic acid library which hybridizes, under stringent hybridization conditions, to a nucleotide sequence having a sequence set forth in SEQ ID NOs: 1, 3, 4, 5, 6, 7, 8, and 9;
(4) a nucleotide sequence which hybridizes under stringent conditions to a nucleotide sequence having a sequence set forth in SEQ ID NOs: 1, 3, 4, 5, 6, 7, 8, and 9;
(5) a nucleotide sequence characterized by at least 80% sequence identity to a nucleotide sequence set forth in SEQ ID NOs: 1, 3, 4, 5, 6, 7, 8, and 9;
(6) a nucleotide sequence characterized by at least 85% sequence identity to a nucleotide sequence set forth in SEQ ID NOs: 1, 3, 4, 5, 6, 7, 8, and 9;
(7) a nucleotide sequence characterized by at least 90% sequence identity to the nucleotide sequence set forth in SEQ ID NOs: 1, 3, 4, 5, 6, 7, 8, and 9;
(8) a nucleotide sequence that comprises the complement of any one of (1), (2), (3), or (4); and
(9) a nucleotide sequence comprising at least 100 contiguous nucleotides from a nucleotide sequence of (1), (2), (3), (4), (5), (6), or (7);
(b) culturing said plant cell under plant cell growing conditions; and
(c) inducing expression of said nucleotide sequence for a time sufficient to modulate the level of said defense induced gene in said plant.

13. The method of claim 12, wherein the plant cell is maize, rice, or wheat.

14. A plant having stably incorporated into its genome at least one nucleotide construct comprising a coding sequence operably linked to a promoter that drives expression of said coding sequence in plant cells, wherein said nucleotide sequence is selected from the group consisting of:
(a) a nucleotide sequence set forth in SEQ ID NO: 1, 3, 4, 5, 6, 7, 8, and 9;
(b) a nucleotide sequence that encodes a polypeptide having an amino acid sequence set forth in SEQ ID NO:2, 10, 11, and 12;
(c) a nucleotide sequence amplified from a maize, rice, or wheat nucleic acid library which hybridizes, under stringent hybridization conditions, to a nucleotide sequence having a sequence set forth in SEQ ID NOs: 1, 3, 4, 5, 6, 7, 8, and 9;
(d) a nucleotide sequence which hybridizes under stringent conditions to a nucleotide sequence having a sequence set forth in SEQ ID NOs: 1, 3, 4, 5, 6, 7, 8, and 9;
(e) a nucleotide sequence characterized by at least 80% sequence identity to a nucleotide sequence set forth in SEQ ID NOs: 1, 3, 4, 5, 6, 7, 8, and 9;
(f) a nucleotide sequence characterized by at least 85% sequence identity to a nucleotide sequence set forth in SEQ ID NOs: 1, 3, 4, 5, 6, 7, 8, and 9;
(g) a nucleotide sequence characterized by at least 90% sequence identity to a nucleotide sequence set forth in SEQ ID NOs: 1, 3, 4, 5, 6, 7, 8, and 9;
(h) a nucleotide sequence that comprises the complement of any one of (a), (b), (c), or (d); and
(i) a nucleotide sequence comprising at least 100 contiguous nucleotides from a nucleotide sequence of (a), (b), (c), (d), (e), (f), or (g).

15. A transformed seed of the plant of claim 14.

16. The plant of claim 14 wherein said plant is a monocot.

17. The plant of claim 14, wherein said plant is a dicot.

18. A plant cell that has been transformed with a DNA construct, said construct comprising a promoter that drives expression in a plant cell operably linked with a nucleotide sequence selected from the group consisting of:
(a) a nucleotide sequence set forth in SEQ ID NO: 1, 3, 4, 5, 6, 7, 8, and 9;
(b) a nucleotide sequence that encodes a polypeptide having an amino acid sequence set forth in SEQ ID NO:2, 10, 11, and 12;
(c) a nucleotide sequence amplified from a maize, rice, or wheat nucleic acid library which hybridizes, under stringent hybridization conditions, to a nucleotide sequence having a sequence set forth in SEQ ID NOs: 1, 3, 4, 5, 6, 7, 8, and 9;
(d) a nucleotide sequence which hybridizes under stringent conditions to a nucleotide sequence having a sequence set forth in SEQ ID NOs: 1, 3, 4, 5, 6, 7, 8, and 9;
(e) a nucleotide sequence characterized by at least 80% sequence identity to a nucleotide sequence set forth in SEQ ID NOs: 1, 3, 4, 5, 6, 7, 8, and 9;
(f) a nucleotide sequence characterized by at least 85% sequence identity to a nucleotide sequence set forth in SEQ ID NOs: 1, 3, 4, 5, 6, 7, 8, and 9;
(g) a nucleotide sequence characterized by at least 90% sequence identity to the nucleotide sequence set forth in SEQ ID NOs: 1, 3, 4, 5, 6, 7, 8, and 9;
(h) a nucleotide sequence that comprises the complement of any one of (a), (b), (c), or (d); and
(i) a nucleotide sequence comprising at least 100 contiguous nucleotides from a nucleotide sequence of (a), (b), (c), (d), (e), (f), or (g).

19. The plant cell of claim 18, wherein said plant cell is a monocot plant cell.

20. The plant cell of claim 18, wherein said plant cell is a dicot plant cell.


SEQUENCE LISTING (83 sequences)

SEQ ID NO: 1 - DNA, 1797 bp, Z. mays
  CDS (109)...(1572)
  5′UTR (1)...(108)
  3′UTR (1576)...(1753)
  polyA_signal (1754)...(1797)
  transit_peptide (109)...(168)
gttttgcatctctctattcattcatccggccaccaccgctctaactactcttcaagagacgacgaccaacaggcacgtcgatcgtcttccggcgagggcgacggaaggatgtccggcggcgagagtggtccggcagcggcggcggcggccgttccgttgctgcaggcgccggaggggaggacgacgaagtactacgagggatgccccgggtgccggctggacgaggccaacaagactaggaccggcgtcccctacctcaatttcttctacatctgggtcgtctgcctcgccgccgcactgccggtccagtcactgttcccttatctatacttcatgatcagggacttgaaagtggcgaaagaggagcaagacattgggttttatgctggttttgttggggctacctatttccttggaagggccatcagcgccgtgccatggggcatgttcgctgacaagtatggaaggaagccatgcattgtgatcagcatcctctcagtgattgtgttcaacacactgtttggacttagcacaacttactggatggcaattgtgactaggggattacttgggttgctatgtggcatacttggacccatcaaggcctatgcttcagaagtctgcaggaaagagcaccaagctctgggaatctctcttgttacatcttcacgagccatagctcttgttattgggcctgctattggaggcttccttgcacagcctgcacagaagtacccaaatcttttctctgaagagtccatatttggaaggtttccatacttccttccttgctttgtaatatcgttgctagcagcaggatcatgtatcgcatgcatttggcttccggaaacgctacactttcatggtgatgacaaagtagaagctattgaagaactggaggcacaagttcgtggctccgaatctacaaaagatctgcataagaattggcaattgatgtcagcaataatcctctactgtgtcttttctatgcatgacacagcttatcttgaggtattttcactgtgggctgtgagcagtagaaaatttcgggggcttagtttgacatcccaggatgttggtactgtgctagccttctcaggttttggtgtacttgtataccaactcgctatttatccttttcttgcgaagtattttggaccaatcaagacatttcggcctgcggcgatcctgtcgatcattctcctcgctacgtatcctttcatggccaatttacatggcctggagcttaaaatactcataaacattgcatctgttttgaagaacatgtttgcggctaccatcactattgcctgcaacatcctacagaacactgcagtgacgcaagagcagagaggcgttgctaatggcatctctgttaccctgatgtccgtgttcaaatctgtagctccagcagcagcaggaattctgttctcgtgggctcagaagcacatcagcggactgttcttaccaggggatcagatcttgttcctagcgataaacatggtgtcggtgaatggcctggtgctgacgttcaagccatttttctccctaccgaatccaacgaggcattcataaatctgtgtagatgaagcccgttcctgtgaattgtatcgatgcactgcctgattacagttgggtttcaaactgcaagaattcatgacttcatgtattgttgtgtggtccaaacttttagtcttatgatgatgaattaggtaatataataataatatatgaagggtttgagctctttcgtaaaaaaaaaaaaaaaaaaaaaaaaaaaaaaaaaaaaaaaaaaaa

SEQ ID NO: 2 - protein, 488 aa, Z. mays
MSGGESGPAAAAAAVPLLQAPEGRTTKYYEGCPGCRLDEANKTRTGVPYLNFFYIWVVCLAAALPVQSLFPYLYFMIRDLKVAKEEQDIGFYAGFVGATYFLGRAISAVPWGMFADKYGRKPCIVISILSVIVFNTLFGLSTTYWMAIVTRGLLGLLCGILGPIKAYASEVCRKEHQALGISLVTSSRAIALVIGPAIGGFLAQPAQKYPNLFSEESIFGRFPYFLPCFVISLLAAGSCIACIWLPETLHFHGDDKVEAIEELEAQVRGSESTKDLHKNWQLMSAIILYCVFSMHDTAYLEVFSLWAVSSRKFRGLSLTSQDVGTVLAFSGFGVLVYQLAIYPFLAKYFGPIKTFRPAAILSIILLATYPFMANLHGLELKILINIASVLKNMFAATITIACNILQNTAVTQEQRGVANGISVTLMSVFKSVAPAAAGILFSWAQKHISGLFLPGDQILFLAINMVSVNGLVLTFKPFFSLPNPTRHS

SEQ ID NO: 3 - DNA, 778 bp, Z. mays
  misc_feature (1)...(778): n = A, T, C, or G
gtgccttcatcatgtatcccttaatcataattgacctctgatgtgtaaaaaagggatagtggtcgacaaaaatatttctagcatcaggaaggtttccataattccttccttgctttgtaatatcgttgctagcagcaggatcatgtattgcatgcatttggcttccggtatgccatatcatcttccctattattttctatcttgaatccaaatagttagatcctcttaagaaaataaaagaaaactctgacaacctatggaggtatgacagctcctgacatagcatgatagagaagaccttctcacctatacggctataccaagaaaagaaccgtgctccatccatacaaacaagatttgttaacccaacataaattcacncctataaaaatttgaactagaaaggctactaaagggcctaaaaacgaaatatgtttggacttatgtttctcccactcaatatacctatttccaagttagataaaaaatcgtccttttgatccaagatagattggaatatacctatttcccacacaccaaaggggccttgggggaccctcctatggaaacacaccattccacacaacggtagtcctgatcaagcgcttgcactttattgcacaatgggctttgggacatcacacgctaacacggggtgtaattctaggcaactcatcgttgcacatttggnaatntcacanattcaancggagcaaactgccggaanttnaatgggntaaggccgancggaatttgnttaaaacgntttgngttnttt

SEQ ID NO: 4 - DNA, 435 bp, Z. mays
  misc_feature (1)...(435): n= A, T, C, or G
attatgtaataggttacatcttcacgagccatagctcttgttattgggcctgctattggaggcttccttgcacaggtgaagattatggaatttagatagcagtataagttaaattcgaaatgctaatgagtgccttcatcatgtatcccttaatcataattgacctctgatgtgtaaaaaagggatagtggtcgacaaaaatatttctagcatcaggaaggtaaattatttctagtagtacaatttctagcatttaactatttcattgtttttagacgactattataaaccttcaaaaattatgcatctaattttcagnttccataattccctcctgctttgtaatatcgttgctagcagcaggatcatgtatgcagcattggcttccggtatgccatatcacttcccnattattgctacttgaatccaaatagt

SEQ ID NO: 5 - DNA, 1656 bp, Z. mays
  misc_feature (1)...(1656): n = A, T, C, or G
attttnngcnngnnnngttggcgcttcatatatgtgtgggagggcagtcttctcaacagtatggggaatcgtggctgataagtatggaaggaaaccagttatcgtactgacccttattgcaatagttatcttcaatactatgtttggactaagctcaaactattggatggcattaatcacacgatgcctgcttggaatcatgtgtggttatctcgggccaattaaggcttatgctacagaagtgtgccgaaaagaatacaatcatctggctttggcagttgtttcttcctcacgaggcattggtctcattattggtccagctattggtggttaccttgcgcaggcctgcagataaatatccaagtatattctctcagacgtccatatttgggaggtttccatactttcttccgtgcctgtgtatatcaatccttgcagttattgctctaattgcctgcatctggtttccggaaactttgcataaacacaatggggatgccgttgataattcagttgaaaccgtagaagaatctcttgctggcaccgacactgaagaaaatggaggtggcggatgtctaaaattatttacaaactggccgttgatgtcagctatnactttatatngtnnctncncnctncngganntggcttangcananacattctctctttgggctgtcagtnananatcgttnggnggantaagctttactacnanagatntgggcaatgtccntgcnatgtcaggtcncntcctttnnctatatcaaatgttnanctntccgnnccntgccaaannggnnnaccanatcacnnnngtnngtnnngnnncntnntngnctctaccggttcttgctagctacccattctttccttcgttgtccgggttcgggctcatggtggtagtaaattgtgcatcttttctgaagaatacattctcggtaactaccattaccgtgttcaacattttgatgaatgaagctgtgactcaggatgtaagagccgcagccaatgggatagctgtaacactaatgtccatctctaaagcggttgctccagctgttgcaggaatcatattttcgtgggcccagaggcgtcaaacagctgcatttcttccaggcgaccacttggtgttcttcatactgaacatcttcacgttcatcggtttcgtcttcaccttcagaccgtttttcgttcgaggcagtgccaagcactgaaacatggatgaaccggacgacgacccatggtatcctatccaggggcaatcgaatataaattttcttccggcgtggccaagggaggtgccgagtctgtaccagtacgacgactcaggccagaacacacggcgtatggtatggtacacagcaaggccgtgttgttgctgtaagttttgtaatttctgtttccttctcggcttcgcagcatgcagttgctgtatatgatggaagaacactctgttactgggcatgttgcgcggtttccacgcgtgtaagtaatgtagaagtcgctatggtaggtatttccataccgtcattagtggatcattgattttttttgtaagaaggttattgcaatgccctttttccgggcatgaataagaaaaaggaaatatgcttatatgcatggtatnaanaaaaaaaaaaaaaaaaaaaaaaa

SEQ ID NO: 6 - DNA, 1640 bp, Z. mays
  misc_feature (1)...(916): n = A, T, C, or G
ccacgcgtccggttgctctgaaatcatccgtgcattttctttctttttatttggcaagggactttccttctcttttctttctcctaatttatagtggtgaccaaacagataagggacctgcgcgtcgctgagagggaggaagacattggattctacgctggtttcctcggcgcagcatacatgatcggcagaggcgtcgcgtcggtcttctggggcgtggtggcggaccgcgttggccggaagcccgtcatcgcgttctccgtcctctcggtcgtggtgttcaacaccctgttcgggctgagcgccaagtactggatggctatcgctacgaggtttctcctgggcgccctcaacggcttccttgcgccagcgaaggcgtactccatcgaggtctgccggcctgagcagcaggctctgggtatatcggtcgtaagcacagcgtgggggatgggcgtcataatcggcccggccatcggtggctacctcgcgcagcctgccaaacagtatccgcacctgttccatgagaaatcagtgtttggaaggttcccgtacctgttgccatgcctgtgcatctcgttcttcgccgccttggttgtcataagctgcgcatggctcccggagaccctacacaagcacagaggcctggagagagcagcagctgaagtggcagaaggcacgacagcagcagcagcagctcaagaaagcaccccggagccggagccggagccacctaagagcagcctgctcaggaaccggccactcatgtcctccatcgtcacgtactgcgtcttctccctccacgacaccgcgtacgtcgagatattttctctgtggaccgtgagtggcagagatcacggcggcctcagcttcgcgtccaaggacgtcggccaagtcctcaccgtcgctggcgccagtctcctggtgtaccaaatcttcgcctaccgttgggtcaacaagatcctcggcccagtcaactcaacccgagtttcgtcggcgctatccataccgataatcgccgcttaccccttcatgacgcgcttgtcgggaataaggcttggtgtgcctctctacgtcgcggcgatgctcaaaagcgtcctcgccatcaccagagtcaccggcacatcgctcctgcagaacaacgctgtgccacaggagcagaggggcgctgcgaacgggatagctacgacggcgatgtctctgtccaaggcgttcgctccggccgtggccggcatcttgttctcgtgggcacagaagcgccagcatgccgcgttctttccaggtgaccagatggtgtttctgctcctgaatctgacggaggtcatcgggctcgtgctcaccttcaagcctttcctcgcggttccccagcagtacaaacagtgaaagcggtgctgcgtgcgtgcgtgctctgcatggatactatagagaggtagctagctgctagccagtgtgtgtgggcgagagcgagacgagggagatggacatgtcaagtagaagcagaggatatgatgtcattatacatatttactttgcgagatatgtagttcctcatgagtcttcgctgtgcggttttctaaaaaaaaattgagataaagttggtctctcaattatatggtgcaaaaaaaaaaaaaaaaaaaaaaaaa

SEQ ID NO: 7 - DNA, 1813 bp, O. sativa
  misc_feature (1)...(473): n = A, T, C, or G
gcacgagggtcagttccacacctctccttcctttaatttctctctgctcgccggagacagactagcttagctagtaggagagggaggaactaatggccgccggagacaaggccggcggcgacgacgctgcggcggcggcggcggcgccattgctggtgtcggcggcggggaggcggcggcggtgccccgggtgcctgacggaggagaggtgcaaggccgacgccggcatcccctacctcaacttcttctacatctgggtcgtctgcctctgctcctcgttgccaattcagtcgttgtttccgtatctgtacttcatgatcagggacttgaaagtcgcaaaggaagagcaagatattgggttctatgccggttttgtcggggctacttatttccttggaagaactatcagtgcagtgccatggggcatttttgctgacaaatatgggaggaagccctgcattgtgatcagtatcctctcagtgattgtgtttaacacactctttggcctcagcacgacttactggatggcaattgttaccaggggattacttggattactctgtggtatactaggaccaatcaaggcctatgcttcagaggtctgcaggaaggagcaccaagctcttggaatttcccttgtcacatcctcgcgagcaatagctctggttgttggaccagctattggaggatttctttcgcagcctgcaaagaaatacccaaatcttttctctgaagaatcggtatttgggaggtttccctactttctcccttgcttcgtcatatcggtactagcagcaggagcatgtgttgcgtgtatttggcttccggaaaccctgcacatgcaccatgatgacaaagaagtcattgatgcactagaggcacaagatgcgacttcagacttaggagaaacaactaaagaatcaggatcagggagaatgggccatacaaagagtttgctgaagaactggcagctgatgtcagcaattaccctctactgtgtcttctctctccatgatacagcttatcttgagatattttcactctgggctgtgagcagcagaaaataccggggcctgagttttacatcccaggatgttggtatcgtgctagctatttccggttttggtgttttggtgtaccaacttgcgatttatccgcttcttgctaaatatgttgggccaatcaagccattccgttatgcagcggtcttgtctatacttctcctttcaacatatccattcatggctaacctgtatggtctggagctcaaagtactcatcaacattgcttcgcttttgaaaaatatgttcgctgctacaattactattgcatgcaacatcctgcaaaacactgcagtgacgcaagaacaaagaggggttgcaaatggaatctctgtcactctgatgtcaatcttcaaagccgtagctccagcagcagctggaattttgttttcatgggcgcaaaagcacattactgggttgttcttaccaggtgagcagatcctgttcctgatgctgaacatggtgtcagtgattgggttcatcctgacattcaagccatttttcgccttgccggatatgcgatgatgtgtagttaggtaacaaaaaggccagaatttatcagacttcagcctggattctaacctgcaagaggaattcatgtactgtaccgcgtgtaatgttcagttgtgtaatcagtttggtggtatctttgatttctgtctgaactccgaaccattggatggtcgaggcatatgataaacagctaacgggatcttgtttcatctaaaaaaaaaaaaaaaaaaaaaaaaaaa

SEQ ID NO: 8 - DNA, 1845 bp, O. sativa
  misc_feature (1)...(1249): n = A, T, C, or G
gccaatctccaccaccacatcatcttcttcctcctccacctcttacctcgtcgtcctgagcgctcctggatgcagttgccttctcctgacaactcctcttcgccttggctaagttagctagctcatcatcacactctgcatacgtgcttgtcaactccattgagagcgtcgccgttgatggctgagccgccggcgaccaaggtgtaccacgatggctgccccggctgcgccatggagcggaggaaggaggagcacaagggcattccctacagggagttcctcttcgttgccatcaccaccctcgcctcctctctgccaatctcttccttgttccccttcctgtacttcatgataagagacttgcatgttgctcggacagaggaagatattggattctatgctggatttcttggcgcatcatatatgatcggtcgtggtttcgcatcgatcttgtggggtatggtggcagatcgtattggccgtaagcctgtcattatcttttccatttttgcagtcattgtgctcaatactttgtttggattaagtgtgaagtactggatggctgttaccacaagatttcttcttggtgctctgaatggattgcttgcaccaataaaggcgtactctatcgaagtttgccgagctgaacatcaacctttgggcctatcaattgtgagcacagcatgggggataggtcttgtagttggcccagcaactgggggatacctcgcacagcctgtcaaacaatatcctcatatttttcatgagaagtcaatatttgggagatttccatatcttctaccctgcctttgtatatcactttttgctctcttggtcctcctaagctgtatatggctaccggagaccctacataagcataaaggccttgaagtgggagtggagacagctgaagcttctactactcaagaaagtgcagaatcacatcagaaaagcttattcagaaattggccattgatgtcatctattgtcacatattgtgtgttctcccttcatgacacagcatatagtgagatattttctctgtggactgtaagtgatagaaaatatggtggactcagcttttcatctaaagatgttgggcaagttcttgcagtggcaggtgccagccttcttgtatatcagctttttatctacggttgggttgataaaattcttggacctatccactcaacccgcatttcagcggcactatctgtaccaattattgctgcttatccctttatgacacacttatcaggaataagacttggtgttgccctctatagtgcagcaatgatcaaaagtgttcttgctataactataattacgggcacctctcttctgcaaaacaaagcagtgccacaagggcaacggggtgctgcaaatggaatagccacaacagccatgtccttgttcaaggctattgctccggctggggcaggagttatattttcttgggcacaaaagagacaacatgtggcattttttccaggtgaccagatggtatttcttctactgaatctgaccgaggtcattgggctcatgttaaccttcaaacctttcttggctgttcctcaacaatataaatagaacattcagatactgctagctggtgtgacaaagatcataaagatgtagttacagtgagtaataagtatggcttggtattaaaagatggtttagatgtggttatagcataatggtaggatcatgcagcattccagtgcagagtctctgttggattttgttctgctttgggcttatgagcaagataaccttgtatattgcagtgttgaatttgaataactgctcttctaaaaaaaaaaaaaaaaaaaaaaaaaaaaa

SEQ ID NO: 9 - DNA, 598 bp, Triticum Spp.
  misc_feature (1)...(598): n = A, T, C, or G
gtcggtccagccattggaggctacttagcacagcctgcaaagcaatatccaaacctattttctgagaattcgatttttggaaggtttccttatttgttgccgtgcctttttatttcactgatcgcctttgctgttctaataagctgcatatggctaccggagacacttcatatgcataaaaacttaagaaagggaagtagaaatggttggtgattcaagagctgctccccatagagaattccacatccaagagaagatctatacaagaactggccgttgatgtcctccataattgcaatgtgtttcacccttcaatgatacagcatacagtgagaatttcccttggnggctgtnaattgaaagattatggcggactaaactttcaacctaaagatgtttngcaantcctgcanttcaaagggctggctcctttgnatcaaatatttgtttataacactccacaatactggggcaacatcacccctatgcaacgcncaacaaacatctgcactacctcaagaacactacaggacaaacggcaacantatccgcgtanaagggcttcacacaactacgcatnttcgaanatgcggtcaaa

SEQ ID NO: 10 - protein, 399 aa, Z. mays
MIGRGVASVFWGVVADRVGRKPVIAFSVLSVVVFNTLFGLSAKYWMAIATRFLLGALNGFLAPAKAYSIEVCRPEQQALGISVVSTAWGMGVIIGPAIGGYLAQPAKQYPHLFHEKSVFGRFPYLLPCLCISFFAALVVISCAWLPETLHKHRGLERAAAEVAEGTTAAAAAQESTPEPEPEPPKSSLLRNRPLMSSIVTYCVFSLHDTAYVEIFSLWTVSGRDHGGLSFASKDVGQVLTVAGASLLVYQIFAYRWVNKILGPVNSTRVSSALSIPIIAAYPFMTRLSGIRLGVPLYVAAMLKSVLAITRVTGTSLLQNNAVPQEQRGAANGIATTAMSLSKAFAPAVAGILFSWAQKRQHAAFFPGDQMVFLLLNLTEVIGLVLTFKPFLAVPQQYKQ

SEQ ID NO: 11 - protein, 497 aa, O. sativa
MAAGDKAGGDDAAAAAAAPLLVSAAGRRRRCPGCLTEERCKADAGIPYLNFFYIWVVCLCSSLPIQSLFPYLYFMIRDLKVAKEEQDIGFYAGFVGATYFLGRTISAVPWGIFADKYGRKPCIVISILSVIVFNTLFGLSTTYWMAIVTRGLLGLLCGILGPIKAYASEVCRKEHQALGISLVTSSRAIALVVGPAIGGFLSQPAKKYPNLFSEESVFGRFPYFLPCFVISVLAAGACVACIWLPETLHMHHDDKEVIDALEAQDATSDLGETTKESGSGRMGHTKSLLKNWQLMSAITLYCVFSLHDTAYLEIFSLWAVSSRKYRGLSFTSQDVGIVLAISGFGVLVYQLAIYPLLAKYVGPIKPFRYAAVLSILLLSTYPFMANLYGLELKVLINIASLLKNMFAATITIACNILQNTAVTQEQRGVANGISVTLMSIFKAVAPAAAGILFSWAQKHITGLFLPGEQILFLMLNMVSVIGFILTFKPFFALPDMR

SEQ ID NO: 12 - protein, 470 aa, O. sativa
MAEPPATKVYHDGCPGCAMERRKEEHKGIPYREFLFVAITTLASSLPISSLFPFLYFMIRDLHVARTEEDIGFYAGFLGASYMIGRGFASILWGMVADRIGRKPVIIFSIFAVIVLNTLFGLSVKYWMAVTTRFLLGALNGLLAPIKAYSIEVCRAEHQPLGLSIVSTAWGIGLVVGPATGGYLAQPVKQYPHIFHEKSIFGRFPYLLPCLCISLFALLVLLSCIWLPETLHKHKGLEVGVETAEASTTQESAESHQKSLFRNWPLMSSIVTYCVFSLHDTAYSEIFSLWTVSDRKYGGLSFSSKDVGQVLAVAGASLLVYQLFIYGWVDKILGPIHSTRISAALSVPIIAAYPFMTHLSGIRLGVALYSAAMIKSVLAITIITGTSLLQNKAVPQGQRGAANGIATTAMSLFKAIAPAGAGVIFSWAQKRQHVAFFPGDQMVFLLLNLTEVIGLMLTFKPFLAVPQQYK

SEQ ID NO: 13 - protein, 10 aa, Z. mays
GMFADKYGRK

SEQ ID NO: 14 - protein, 19 aa, Z. mays
SLVTSSRAIALVIGPAIGG

SEQ ID NO: 15 - protein, 12 aa, Z. mays
AKYFGPIKTFRP

SEQ ID NO: 16 - protein, 8 aa, Z. mays
FSMHDTAY

SEQ ID NO: 17 - protein, 10 aa, O. sativa
GIFADKYGRK

SEQ ID NO: 18 - protein, 19 aa, O. sativa
SLVTSSRAIALVVGPAIGG

SEQ ID NO: 19 - protein, 12 aa, O. sativa
AKYVGPIKPFRY

SEQ ID NO: 20 - protein, 8 aa, O. sativa
FSLHDTAY

SEQ ID NO: 21 - protein, 19 aa, O. sativa
SLVTSSRAIALVVGPAIGG

SEQ ID NO: 22 - protein, 12 aa, O. sativa
AKYVGPIKPFRY

SEQ ID NO: 23 - protein, 8 aa, O. sativa
FSLHDTAY

SEQ ID NO: 24 - protein, 10 aa, O. sativa
GIVADKYGRK

SEQ ID NO: 25 - protein, 19 aa, O. sativa
SLVSSSRGIGLIVGPAIGG

SEQ ID NO: 26 - protein, 12 aa, O. sativa
AKSVEPITLVRI

SEQ ID NO: 27 - protein, 8 aa, O. sativa
FSLQDVAY

SEQ ID NO: 28 - protein, 10 aa, O. sativa
GMVADRIGRK

SEQ ID NO: 29 - protein, 19 aa, O. sativa
SIVSTAWGIGLVVGPATGG

SEQ ID NO: 30 - protein, 12 aa, O. sativa
DKILGPIHSTRI

SEQ ID NO: 31 - protein, 8 aa, O. sativa
FSLHDTAY

SEQ ID NO: 32 - protein, 10 aa, Z. mays
GVVADRVGRK

SEQ ID NO: 33 - protein, 19 aa, Z. mays
SVVSTAWGMGVIIGPAIGG

SEQ ID NO: 34 - protein, 12 aa, Z. mays
NKILGPVNSTRV

SEQ ID NO: 35 - protein, 8 aa, Z. mays
FSLHDTAY

SEQ ID NO: 36 - protein, 10 aa, A. thaliana
GKLADRYGRK

SEQ ID NO: 37 - protein, 19 aa, A. thaliana
SVVSTSRGIGLILGPAIGG

SEQ ID NO: 38 - protein, 12 aa, A. thaliana
EKSVGLLAVIRL

SEQ ID NO: 39 - protein, 8 aa, A. thaliana
FSLQEIAY

SEQ ID NO: 40 - protein, 10 aa, A. thaliana
GLVADRYGRK

SEQ ID NO: 41 - protein, 19 aa, A. thaliana
SAVSTAWGIGLIIGPAIGG

SEQ ID NO: 42 - protein, 12 aa, A. thaliana
ERLLGPIIVTRI

SEQ ID NO: 43 - protein, 8 aa, A. thaliana
FSLHDMAY

SEQ ID NO: 44 - protein, 10 aa, A. thaliana
GIVADRYGRK

SEQ ID NO: 45 - protein, 19 aa, A. thaliana
SAVSTAWGIGLIIGPALGG

SEQ ID NO: 46 - protein, 12 aa, A. thaliana
EKLLGPVLVTRY

SEQ ID NO: 47 - protein, 8 aa, A. thaliana
LCLHDTAY

SEQ ID NO: 48 - protein, 10 aa, E. coli
GKMSDRFGRR

SEQ ID NO: 49 - protein, 19 aa, E. coli
GWLGASFGLGLIAGPIIGG

SEQ ID NO: 50 - protein, 10 aa, E. coli
GRIATKWGEK

SEQ ID NO: 51 - protein, 8 aa, E. coli
AQLIGQIP

SEQ ID NO: 52 - protein, 10 aa, S. aureus
GTLADKLGKK

SEQ ID NO: 53 - protein, 19 aa, S. aureus
GYMSAIINSGFILGPGIGG

SEQ ID NO: 54 - protein, 10 aa, S. aureus
DKFMKYFSEL

SEQ ID NO: 55 - protein, 8 aa, S. aureus
LAFGLSAF

SEQ ID NO: 56 - protein, 10 aa, B. subtilis
GRWVDRFGRK

SEQ ID NO: 57 - protein, 19 aa, B. subtilis
GYVSAAISTGFIIGPGAGG

SEQ ID NO: 58 - protein, 10 aa, B. subtilis
GKLVNKLGEK

SEQ ID NO: 59 - protein, 8 aa, B. subtilis
MAFGLSAY

SEQ ID NO: 60 - protein, 10 aa, S. pneumoniae
GILADKYGRK

SEQ ID NO: 61 - protein, 19 aa, S. pneumoniae
GTLSTGVVAGTLTGPFIGG

SEQ ID NO: 62 - protein, 10 aa, S. pneumoniae
GKLGDKVGNH

SEQ ID NO: 63 - protein, 8 aa, S. pneumoniae
IQFSAQSI

SEQ ID NO: 64 - protein, 10 aa, E. coli
GGLADRKGRK

SEQ ID NO: 65 - protein, 19 aa, E. coli
GTLSTGGVSGALLGPMAGG

SEQ ID NO: 66 - protein, 10 aa, E. coli
GKLGDRIGPE

SEQ ID NO: 67 - protein, 8 aa, E. coli
IQVATGSI

SEQ ID NO: 68 - protein, 10 aa, S. cerevisiae
GRFSEKHGRK

SEQ ID NO: 69 - protein, 19 aa, S. cerevisiae
STMPLLFQFGAVVGPMIGG

SEQ ID NO: 70 - protein, 12 aa, S. cerevisiae
DRNFDCLTIFRT

SEQ ID NO: 71 - protein, 8 aa, S. cerevisiae
MALHLIVY

SEQ ID NO: 72 - protein, 10 aa, B. subtilis
GRWVDRFGRK

SEQ ID NO: 73 - protein, 19 aa, B. subtilis
GYMSAAISTGFIIGPGIGG

SEQ ID NO: 74 - protein, 10 aa, B. subtilis
DRFTRWFGEI

SEQ ID NO: 75 - protein, 8 aa, B. subtilis
SSFGLASF

SEQ ID NO: 76 - protein, 10 aa, P. mirabilis
GKLSDKYGRK

SEQ ID NO: 77 - protein, 19 aa, P. mirabilis
GFLGGAFGVGLIIGPMLGG

SEQ ID NO: 78 - protein, 10 aa, P. mirabilis
GKLAQKWGER

SEQ ID NO: 79 - protein, 8 aa, P. mirabilis
IQLIGQIP

SEQ ID NO: 80 - protein, 10 aa, A. tumefaciens
GALSDRFGRR

SEQ ID NO: 81 - protein, 19 aa, A. tumefaciens
GTVGAVMSLGFIIGPVIGG

SEQ ID NO: 82 - protein, 10 aa, A. tumefaciens
GPLSRRFGDL

SEQ ID NO: 83 - protein, 8 aa, A. tumefaciens
FGLVAAIP